SEQUENCE LISTING (67 sequences)

SEQ ID NO: 1 - DNA, 168 bp, Corynebacterium glutamicum
cgattccccatgatgccctaacatcttgcaggtgaggggtacatattggggcaattcgggggtaattttgcagtatcgtcaagatcacccaaaactggtggctgttctcttttaagcgggatagcatgggttcttagaggaccccctacaaggattgaggattgttta

SEQ ID NO: 2 - DNA, 180 bp, Streptococcus mutans
atgacaaaacaatataaaaattatgtcaatggcgagtggaagctttcagaaaatgaaattaaaatctacgaaccggccagtggagctgaattgggttcagttccagcaatgagtactgaagaagtagattatgtttatgcttcagccaagaaagctcaaccagcttggcgatcactttca

SEQ ID NO: 3 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgcaggtaacaactgcgagtacggtgggttcttagaggaccccctacaaggattgaggattgttta

SEQ ID NO: 4 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgagtctgagggatataattaagatgggttcttagaggaccccctacaaggattgaggattgttta

SEQ ID NO: 5 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgggtcgtggaggacaagttaagatgggttcttagaggaccccctacaaggattgaggattgttta

SEQ ID NO: 6 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgctactaccatcgggtgctaaggtgggttcttagaggaccccctacaaggattgaggattgttta

SEQ ID NO: 7 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctggataggtaggcgttaagtaaactgggttcttagaggaccccctacaaggattgaggattgttta

SEQ ID NO: 8 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgggcaattcgcggggaggtagactgggttcttagaggaccccctacaaggattgaggattgttta

SEQ ID NO: 9 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgtattgggcgcggcattataggatgggttcttagaggaccccctacaaggattgaggattgttta

SEQ ID NO: 10 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctggggaactgctttggtagtatcatgggttcttagaggaccccctacaaggattgaggattgttta

SEQ ID NO: 11 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctggttcaggtttgggccgctatgctgggttcttagaggaccccctacaaggattgaggattgttta

SEQ ID NO: 12 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgtgatcggggcgctttggtacggtgggttcttagaggaccccctacaaggattgaggattgttta

SEQ ID NO: 13 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgtatgaaataaattggagtaggctgggttcttagaggaccccctacaaggattgaggattgttta

SEQ ID NO: 14 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgaacggtatctttctccatataatgggttcttagaggaccccctacaaggattgaggattgttta

SEQ ID NO: 15 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgaacggagcgtgtacgactagaatgggttcttagaggaccccctacaaggattgaggattgttta

SEQ ID NO: 16 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgtgcgcgggtaaacgctgtagagtgggttcttagaggaccccctacaaggattgaggattgttta

SEQ ID NO: 17 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgttgatgtcgtagcgtagtattatgggttcttagaggaccccctacaaggattgaggattgttta

SEQ ID NO: 18 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgagctcggactttggtggtaggctgggttcttagaggaccccctacaaggattgaggattgttta

SEQ ID NO: 19 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgccggcgcattataacgctacaatgggttcttagaggaccccctacaaggattgaggattgttta

SEQ ID NO: 20 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgcgggggtgtaagtcgagtagactgggttcttagaggaccccctacaaggattgaggattgttta

SEQ ID NO: 21 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgacgggtaggtatttcgctaggatgggttcttagaggaccccctacaaggattgaggattgttta

SEQ ID NO: 22 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgtagtgtatgcttttacatagactgggttcttagaggaccccctacaaggattgaggattgttta

SEQ ID NO: 23 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgaggcagcatgtacatgatatcgtgggttcttagaggaccccctacaaggattgaggattgttta

SEQ ID NO: 24 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgttgagcggagttaatagtacactgggttcttagaggaccccctacaaggattgaggattgttta

SEQ ID NO: 25 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgatacacacaactttcgatactatgggttcttagaggaccccctacaaggattgaggattgttta

SEQ ID NO: 26 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgattccaaccttatgttatactgtgggttcttagaggaccccctacaaggattgaggattgttta

SEQ ID NO: 27 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctggaggtagtcgtttggggtaggatgggttcttagaggaccccctacaaggattgaggattgttta

SEQ ID NO: 28 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgattgctcgcgtaaatggtaaactgggttcttagaggaccccctacaaggattgaggattgttta

SEQ ID NO: 29 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgtgttattatgggagatgtaagttgggttcttagaggaccccctacaaggattgaggattgttta

SEQ ID NO: 30 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctggaaaaatgtacgtgtgttaaaatgggttcttagaggaccccctacaaggattgaggattgttta

SEQ ID NO: 31 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgagtcctcagtggagtggtaacatgggttcttagaggaccccctacaaggattgaggattgttta

SEQ ID NO: 32 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgtttccggtgttttcgcgtagagtgggttcttagaggaccccctacaaggattgaggattgttta

SEQ ID NO: 33 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgggcgtcggtaggtatgttattgtgggttcttagaggaccccctacaaggattgaggattgttta

SEQ ID NO: 34 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgacggcgatcagagtgtgtaagatgggttcttagaggaccccctacaaggattgaggattgttta

SEQ ID NO: 35 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgccgtgcgtgatatggggtagtgtgggttcttagaggaccccctacaaggattgaggattgttta

SEQ ID NO: 36 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctggggttgtgagttatcggtataatgggttcttagaggaccccctacaaggattgaggattgttta

SEQ ID NO: 37 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgtgctccgatgatggtggtaagatgggttcttagaggaccccctacaaggattgaggattgttta

SEQ ID NO: 38 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctggttctcacgggtccgtgtatgatgggttcttagaggaccccctacaaggattgaggattgttta

SEQ ID NO: 39 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgtttcggaatggatggtgtacgctgggttcttagaggaccccctacaaggattgaggattgttta

SEQ ID NO: 40 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgattatgtcttcttttggtaaaatgggttcttagaggaccccctacaaggattgaggattgttta

SEQ ID NO: 41 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctggatcataggttctgtggtagattgggttcttagaggaccccctacaaggattgaggattgttta

SEQ ID NO: 42 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgcatggccgatcatgatgtaatatgggttcttagaggaccccctacaaggattgaggattgttta

SEQ ID NO: 43 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgtagtggcggtgtacgtgtatgatgggttcttagaggaccccctacaaggattgaggattgttta

SEQ ID NO: 44 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgatttgttacaggtatggtagtttgggttcttagaggaccccctacaaggattgaggattgttta

SEQ ID NO: 45 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgacggaacttatttctggtaacgtgggttcttagaggaccccctacaaggattgaggattgttta

SEQ ID NO: 46 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgaccgggaggactaatggtatggtgggttcttagaggaccccctacaaggattgaggattgttta

SEQ ID NO: 47 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctggccacccctttttctgctattatgggttcttagaggaccccctacaaggattgaggattgttta

SEQ ID NO: 48 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgttgcgccggagttgcggtatagtgggttcttagaggaccccctacaaggattgaggattgttta

SEQ ID NO: 49 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctggtttacgggtgttcatgtagtatgggttcttagaggaccccctacaaggattgaggattgttta

SEQ ID NO: 50 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgtttgtctcgtatctgtgtaagatgggttcttagaggaccccctacaaggattgaggattgttta

SEQ ID NO: 51 - DNA, 168 bp, Artificial Sequence
  sequecne of mdh mutant
cgattccccatgatgccctaacatcttgcaggtgaggggtacatattggggcaattcgggggtaattttgcagtatcgtcaagatcacccaaaactggtggctgattttgggcacttagtgtattgtgggttcttagaggaccccctacaaggattgaggattgttta

SEQ ID NO: 52 - DNA, 42 bp, Artificial Sequence
  primer sequence
cctgatgcggtattttctcccgattccccatgatgccctaac

SEQ ID NO: 53 - DNA, 24 bp, Artificial Sequence
  primer sequence
taaacaatcctcaatccttgtagg

SEQ ID NO: 54 - DNA, 47 bp, Artificial Sequence
  primer sequence
caaggattgaggattgtttaatgacaaaacaatataaaaattatgtc

SEQ ID NO: 55 - DNA, 41 bp, Artificial Sequence
  primer sequence
ccacctccagagccaccgcctgaaagtgatcgccaagctgg

SEQ ID NO: 56 - DNA, 20 bp, Artificial Sequence
  primer sequence
ctgcaggcatgcaagcttgg

SEQ ID NO: 57 - DNA, 21 bp, Artificial Sequence
  primer sequence
ggagaaaataccgcatcaggc

SEQ ID NO: 58 - DNA, 64 bp, Artificial Sequence
  primer sequence
ggcggtggctctggaggtggtgggtccggcggtggctctgcttcctccgaagacgttatcaaag

SEQ ID NO: 59 - DNA, 43 bp, Artificial Sequence
  primer sequence
ccaagcttgcatgcctgcagttaagcaccggtggagtgacgac

SEQ ID NO: 60 - DNA, 65 bp, Artificial Sequence
  primer sequence
  misc_feature (21)..(37): n is a, c, g, or t
  misc_feature (40)..(42): n is a, c, g, or t
tcacccaaaactggtggctgnnnnnnnnnnnnnnnnntannntgggttcttagaggaccccctac

SEQ ID NO: 61 - DNA, 22 bp, Artificial Sequence
  primer sequence
cagccaccagttttgggtgatc

SEQ ID NO: 62 - DNA, 25 bp, Artificial Sequence
  primer sequence
aaccttccatacgaactttgaaacg

SEQ ID NO: 63 - DNA, 23 bp, Artificial Sequence
  primer sequence
caaagttcgtatggaaggttccg

SEQ ID NO: 64 - DNA, 42 bp, Artificial Sequence; identical to SEQ ID NO: 52
  primer sequence

SEQ ID NO: 65 - DNA, 44 bp, Artificial Sequence
  primer sequence
tcgcgtaaaaaatcagtcattaaacaatcctcaatccttgtagg

SEQ ID NO: 66 - DNA, 24 bp, Artificial Sequence
  primer sequence
atgactgattttttacgcgatgac

SEQ ID NO: 67 - DNA, 39 bp, Artificial Sequence
  primer sequence
ccaagcttgcatgcctgcagctagccggagttgcgcagc

(19) United States
(12) Patent Application Publication
(10) Pub. No.: US 2024/0084314 A1
(43) Pub. Date: Mar. 14, 2024

(54) MDH GENE-BASED POLYNUCLEOTIDE HAVING PROMOTER ACTIVITY AND USE THEREOF

(71) Applicant: TIANJIN INSTITUTE OF INDUSTRIAL BIOTECHNOLOGY, CHINESE ACADEMY OF SCIENCES, Tianjin (CN)

(72) Inventors: Ping Zheng, Tianjin (CN); Jiao Liu, Tianjin (CN); Jibin Sun, Tianjin (CN); Guannan Sun, Tianjin (CN); Wenjuan Zhou, Tianjin (CN); Xuan Guo, Tianjin (CN)

(21) Appl. No.: 18/269,046

(22) PCT Filed: May 24, 2022
(86) PCT No.: PCT/CN2022/094736

§ 371 Date: Jun. 22, 2023

(30) Foreign Application Priority Data
Jun. 8, 2021 (CN) 202110638496.5

(51) Int. Cl. C12N 15/77; C12N 15/52; C12N 15/67; C12P 13/04

(52) U.S. Cl. CPC C12N 15/77; C12N 15/52; C12N 15/67; C12P 13/04; C12R 2001/15

(57) ABSTRACT

A polynucleotide having promoter activity of a malate dehydrogenase gene (mdh gene), a transcription expression cassette, recombinant expression vector and recombinant host cell containing the polynucleotide having the promoter activity, a method for constructing a promoter mutant, a method for regulating the transcription of a target gene, a method for preparing a protein, and method for producing a target compound. The polynucleotide having the promoter activity is a mutant of an mdh gene promoter, and compared with a promoter of a wild-type mdh gene, the promoter activity of the mutant is significantly improved. After operably linking the mutant to a target gene, the expression efficiency of the target gene can be significantly improved, thereby effectively improving the yield and transformation rate of a target compound.

### TECHNICAL FIELD

The present disclosure pertains to the field of biotechnology and genetic engineering. and particularly relates to a polynucleotide having promoter activity; and a transcription expression cassette, a recombinant expression vector and a recombinant host cell comprising the polynucleotide having promoter activity, as well as a method for constructing a promoter mutant, a method for regulating transcription of a target gene, and a method for preparing a protein and a method for producing a target compound.

### BACKGROUND

Microbial fermentation can produce a variety of target compounds, such as amino acids and organic acids, which can be widely used in medicine, food, animal feed and cosmetics, and have great economic value. In recent years, with the increasing market demand for amino acids, organic acids and so on, how to increase the output of a target compound and realize industrial large-scale production of the target compound is an important problem to be solved urgently.

Breeding high-yield fermentation microorganisms is an important means to improve the industrial yield of target compounds. Compared with traditional mutation breeding technology, genetic engineering breeding technology has been widely used because of its strong pertinence and high efficiency. Numerous studies have shown that the efficient expression of a key gene in the synthesis pathway of a target compound is the key to improve the yield and conversion rate of the target compound.

It is an important method to improve the fermentation yield of a target compound by transforming a key gene in the microbial metabolic pathway through genetic engineering. Promoter is an important regulatory element that affects gene expression, and fine regulation of the promoter can optimize the conversion rate of the target compound. Promoters with different expression intensities can meet the needs of different expression intensities of different genes, thereby improving the yield and conversion rate of the target compound.

Therefore, it is an important problem to be solved urgently in the field of microbial fermentation to develop more promoters with high activity so as to enhance the expression of key genes in the synthesis pathway of target compounds, improve the yield of target compounds and enhance the competitiveness of bio-fermentation industry.

### SUMMARY

### Technical problem

In view of the technical problem existing in the prior art, for example, it is necessary to develop more promoters with high activity so as to enhance the expression of key genes in the synthesis pathway of target compounds, the present disclosure provides a polynucleotide having promoter activity, which is a mutant of the polynucleotide comprising the sequence as set forth in SEQ ID NO: 1. The mutant provided by the present disclosure has promoter activity significantly improved as compared with the wild-type promoter, and provides an expression regulatory element with great application potential for the transformation of a target gene. By operably ligating the mutant with the target gene, the expression of the target gene can be effectively improved, thereby effectively improving the yield and conversion rate of a target compound.

### Solution to problem

The present disclosure provides a polynucleotide having promoter activity, wherein the polynucleotide is selected from the group shown in any one of the following (i)-(iv):
(i) a mutant of a polynucleotide comprising the sequence as set forth in SEQ ID NO: 1, comprising a mutated nucleotide at one or more positions in position 105 to position 126 of the sequence as set forth in SEQ ID NO: 1;
(ii) a polynucleotide comprising a sequence complementary to the nucleotide sequence as set forth in (i);
(iii) a polynucleotide comprising a reversely complementary sequence of a sequence capable of hybridizing with the nucleotide sequence as set forth in (i) or (ii) under high stringent hybridization conditions or very high stringent hybridization conditions;
(iv) a polynucleotide having at least 90%, optionally at least 95%, preferably at least 97%, more preferably at least 98%, and most preferably at least 99% sequence identity compared with the nucleotide sequence as set forth in (i) or (ii);

wherein the polynucleotide as set forth in any one of (i) to (iv) has a nucleotide sequence that is not TTCTCTTTTAAGCGGGATAGCA at position 105 to position 126 of the sequence as set forth in SEQ ID NO: 1; and the polynucleotide as set forth in any one of (i) to (iv) has enhanced promoter activity compared with the polynucleotide having the sequence as set forth in SEQ ID NO: 1.

In some embodiments, according to the polynucleotide having promoter activity of the present disclosure, wherein the mutant has 0.4 to 13 folds or more improved promoter activity as compared with the promoter activity of the polynucleotide having the sequence as set forth in SEQ ID NO: 1.

In some embodiments, according to the polynucleotide having promoter activity of the present disclosure, wherein the nucleotide sequence of the mutant corresponding to position 105 to position 126 of the sequence as set forth in SEQ ID NO: 1 is selected from any one of the group consisting of the following (P_{mdh}-1) to (P_{mdh}-49):

(P_{mdh}-1)
CAGGTAACAACTGCGAGTACGG;
(P_{mdh}-2)
AGTCTGAGGGATATAATTAAGA;
(P_{mdh}-3)
GGTCGTGGAGGACAAGTTAAGA;
(P_{mdh}-4)
CTACTACCATCGGGTGCTAAGG;
(P_{mdh}-5)
GATAGGTAGGCGTTAAGTAAAC;
(P_{mdh}-6)
GGCAATTCGCGGGGAGGTAGAC;
(P_{mdh}-7)
TATTGGGCGCGGCATTATAGGA;
(P_{mdh}-8)
GGGAACTGCTTTGGTAGTATCA;
(P_{mdh}-9)
GTTCAGGTTTGGGCCGCTATGC;
(P_{mdh}-10)
TGATCGGGGCGCTTTGGTACGG;
(P_{mdh}-11)
TATGAAATAAATTGGAGTAGGC;
(P_{mdh}-12)
AACGGTATCTTTCTCCATATAA;
(P_{mdh}-13)
AACGGAGCGTGTACGACTAGAA;
(P_{mdh}-14)
TGCGCGGGTAAACGCTGTAGAG;
(P_{mdh}-15)
TTGATGTCGTAGCGTAGTATTA;
(P_{mdh}-16)
AGCTCGGACTTTGGTGGTAGGC;
(P_{mdh}-17)
CCGGCGCATTATAACGCTACAA;
(P_{mdh}-18)
CGGGGGTGTAAGTCGAGTAGAC;
(P_{mdh}-19)
ACGGGTAGGTATTTCGCTAGGA;
(P_{mdh}-20)
TAGTGTATGCTTTTACATAGAC;
(P_{mdh}-21)
AGGCAGCATGTACATGATATCG;
(P_{mdh}-22)
TTGAGCGGAGTTAATAGTACAC;
(P_{mdh}-23)
ATACACACAACTTTCGATACTA;
(P_{mdh}-24)
ATTCCAACCTTATGTTATACTG;
(P_{mdh}-25)
GAGGTAGTCGTTTGGGGTAGGA;
(P_{mdh}-26)
ATTGCTCGCGTAAATGGTAAAC;
(P_{mdh}-27)
TGTTATTATGGGAGATGTAAGT;
(P_{mdh}-28)
GAAAAATGTACGTGTGTTAAAA;
(P_{mdh}-29)
AGTCCTCAGTGGAGTGGTAACA;
(P_{mdh}-30)
TTTCCGGTGTTTTCGCGTAGAG;
(P_{mdh}-31)
GGCGTCGGTAGGTATGTTATTG;
(P_{mdh}-32)
ACGGCGATCAGAGTGTGTAAGA;
(P_{mdh}-33)
CCGTGCGTGATATGGGGTAGTG;
(P_{mdh}-34)
GGGTTGTGAGTTATCGGTATAA;
(P_{mdh}-35)
TGCTCCGATGATGGTGGTAAGA;
(P_{mdh}-36)
GTTCTCACGGGTCCGTGTATGA;
(P_{mdh}-37)
TTTCGGAATGGATGGTGTACGC;
(P_{mdh}-38)
ATTATGTCTTCTTTTGGTAAAA;
(P_{mdh}-39)
GATCATAGGTTCTGTGGTAGAT;
(P_{mdh}-40)
CATGGCCGATCATGATGTAATA;
(P_{mdh}-41)
TAGTGGCGGTGTACGTGTATGA;
(P_{mdh}-42)
ATTTGTTACAGGTATGGTAGTT;
(P_{mdh}-43)
ACGGAACTTATTTCTGGTAACG;
(P_{mdh}-44)
ACCGGGAGGACTAATGGTATGG;
(P_{mdh}-45)
GCCACCCCTTTTTCTGCTATTA;
(P_{mdh}-46)
TTGCGCCGGAGTTGCGGTATAG;
(P_{mdh}-47)
GTTTACGGGTGTTCATGTAGTA;
(P_{mdh}-48)
TTTGTCTCGTATCTGTGTAAGA;
(P_{mdh}-49)
ATTTTGGGCACTTAGTGTATTG.

In some embodiments, according to the polynucleotide having promoter activity of the present disclosure, wherein the nucleotide sequence of the mutant is selected from sequences as set forth in any one of SEQ ID NOs: 3 to 51.

The present disclosure further provides a transcription expression cassette, comprising the polynucleotide having promoter activity according to the present disclosure; optionally, the transcription expression cassette further contains a target gene that is operably ligated to the polynucleotide having promoter activity; preferably, the target gene is a protein coding gene.

The present disclosure further provides a recombinant expression vector comprising the polynucleotide having promoter activity according to the present disclosure, or the transcription expression cassette of the present disclosure.

The present disclosure further provides a recombinant host cell comprising the transcription expression cassette of the present disclosure, or the recombinant expression vector of the present disclosure.

In some embodiments, according to the recombinant host cell of the present disclosure, wherein the host cell is from genus Corynebacterium, genus Brevibacterium, genus Arthrobacterium, genus Microbacterium or genus Escherichia; preferably, the host cell is Corynebacterium glutamicum or Escherichia coli; more preferably, the host cell is Corynebacterium glutamicum ATCC 13032, Corynebacterium glutamicum ATCC 13869, Corynebacterium glutamicum ATCC 14067 or derivative strains of Corynebacterium glutamicum.

The present disclosure further provides the use of the polynucleotide having promoter activity according to the present disclosure, the transcription expression cassette according to the present disclosure, the recombinant expression vector according to the present disclosure, and the recombinant host cell according to the present disclosure in at least one of the following:
a) enhancing the transcription level of a gene, or preparing a reagent or kit for enhancing the transcription level of a gene;
b) preparing a protein, or preparing a reagent or kit for preparing a protein;
c) producing a target compound, or preparing a reagent or kit for producing a target compound.

In some embodiments, according to the use of the present disclosure, wherein the protein is selected from a gene expression regulatory protein, a protein related to the synthesis of a target compound, or a protein related to membrane transport.

In some embodiments, according to the use of the present disclosure, wherein the target compound includes at least one of amino acids, organic acids;

optionally; the amino acids include one or a combination of two or more of the following: proline, hydroxyproline, lysine, glutamic acid, threonine, glycine, alanine, valine, leucine, isoleucine, serine, cysteine, glutamine, methionine, aspartic acid, asparagine, arginine, histidine, phenylalanine, tyrosine, tryptophan, 5-aminolevulinic acid or derivatives of any one of the above amino acids;

optionally; the organic acids include one or a combination of two or more of the following: citric acid, succinic acid, lactic acid, acetic acid, butyric acid, palmitic acid, oxalic acid, oxaloacetic acid, tartaric acid, propionic acid, hexenoic acid, decanoic acid, caprylic acid, valeric acid, malic acid or derivatives of any one of the above amino acids.

The present disclosure further provides a construction method of a promoter mutant, including the following steps:

Mutation step: mutating a polynucleotide having the sequence as set forth in SEQ ID NO: 1 so that there are mutated nucleotides at one or more positions in position 105 to position 126 of the sequence as set forth in SEQ ID NO: 1;

Screening step: screening for a mutant of a polynucleotide having improved promoter activity as compared with the polynucleotide having the sequence as set forth in SEQ ID NO: 1, to obtain a promoter mutant.

In some embodiments, according to the construction method of the present disclosure, wherein the mutation step includes:

mutating the polynucleotide having the sequence as set forth in SEQ ID NO: 1 so that the nucleotides at position 105 to position 126 of the sequence as set forth in SEQ ID NO: 1 are mutated to a nucleotide sequence as set forth below: NNNNNNNNNNNNNNNNNTANNNT; wherein N is selected from A, T, C or G;

preferably; the promoter mutant has 0.4 to 13 folds or more improved promoter activity as compared with the promoter activity of the polynucleotide having the sequence as set forth in SEQ ID NO: 1.

The present disclosure further provides a method for regulating transcription, wherein the method includes a step of operably ligating the polynucleotide having promoter activity of the present disclosure to a target RNA or a target gene. Optionally; the target RNA includes at least one of tRNA, sRNA, the target gene includes at least one of a coding gene of a protein related to the synthesis of a target compound, a coding gene of a gene expression regulatory protein and a coding gene of a protein related to membrane transport:

optionally, the target gene includes at least one of the following genes: pyruvate carboxylase gene, phosphoenolpyruvate carboxylase gene, γ-glutamyl kinase gene, glutamate semialdehyde dehydrogenase gene, pyrroline-5-carboxylate reductase gene, amino acid transport protein gene, ptsG system related gene, pyruvate dehydrogenase gene, homoserine dehydrogenase gene, oxaloacetic acid decarboxylase gene, gluconic acid repressor gene, glucose dehydrogenase gene, aspartate kinase gene, aspartate semialdehyde dehydrogenase gene, aspartate ammonia lyase gene, dihydrodipicolinate synthase gene, dihydropyridinic acid reductase gene, succinyl diaminopimelate aminotransferase gene, tetrahydrodipicolinate succinylase gene, succinyl diaminopimelate deacylase gene, diaminopimelic acid epimerase gene, diaminopimelic acid deacylase gene, glyceraldehyde-3-phosphate dehydrogenase gene, transketolase gene, diaminopimelate dehydrogenase gene.

The present disclosure further provides a method for preparing a protein, wherein the method includes a step of expressing the protein by using the transcription expression cassette of the present disclosure, the recombinant expression vector of the present disclosure, or the recombinant host cell of the present disclosure: optionally, the protein is a protein related to the synthesis of a target compound, a protein related to membrane transport, or a gene expression regulatory protein;

optionally, the method further includes a step of isolating or purifying the protein.

The present disclosure further provides a method for producing a target compound, wherein the method includes a step of expressing a protein related to the synthesis of a target compound, a protein related to membrane transport, or a gene expression regulatory protein by using the transcription expression cassette of the present disclosure, the recombinant expression vector of the present disclosure, or the recombinant host cell of the present disclosure, and producing the target compound in the presence of the protein associated with the synthesis of the target compound or the gene expression regulatory protein:

optionally: the target compound includes at least one of amino acids, organic acids:

optionally: the amino acids include one or a combination of two or more of the following: lysine, glutamic acid, threonine, proline, hydroxyproline, glycine, alanine, valine, leucine, isoleucine, serine, cysteine, glutamine, methionine, aspartic acid, asparagine, arginine, histidine, phenylalanine, tyrosine, tryptophan, 5-aminolevulinic acid or derivatives of any one of the above amino acids;

optionally; the organic acids include one or a combination of two or more of the following: citric acid, succinic acid, lactic acid, acetic acid, butyric acid, palmitic acid, oxalic acid, oxaloacetic acid, tartaric acid, propionic acid, hexenoic acid, decanoic acid, caprylic acid, valeric acid, malic acid or derivatives of any one of the above organic acids;

optionally; the protein related to the synthesis of the target compound is a protein related to the synthesis of an L-amino acid; optionally; the protein related to the synthesis of an L-amino acid includes one or a combination of two or more of: pyruvate carboxylase, phosphoenolpyruvate carboxylase, γ-glutamyl kinase, glutamate semialdehyde dehydrogenase, pyrroline-5-carboxylate reductase, amino acid transport protein, ptsG system, pyruvate dehydrogenase, homoserine dehydrogenase, oxaloacetic acid decarboxylase, gluconic acid repressor, glucose dehydrogenase, aspartate kinase, aspartate semialdehyde dehydrogenase, aspartate ammonia lyase, dihydrodipicolinate synthase, dihydropyridinic acid reductase, succinyl diaminopimelate aminotransferase, tetrahydrodipicolinate succinylase, succinyl diaminopimelate deacylase, diaminopimelic acid epimerase, diaminopimelic acid deacylase, glyceraldehyde-3-phosphate dehydrogenase, transketolase, diaminopimelate dehydrogenase.

optionally; the method further includes a step of isolating or purifying the target compound.

### Effects

In some embodiments, the polynucleotide having promoter activity provided by the present disclosure is a mutant of malate dehydrogenase gene (mdh gene) promoter, and the mutant has a promoter activity significantly improved as compared with the wild-type mdh gene. After the mutant is operably ligated to a target gene, the expression efficiency of the target gene can be significantly improved, thereby providing an expression element having great application potential for the modification of a key gene in the synthesis pathway of a target compound. Where the mutant is applied to the production of a target compound, the conversion rate of the target compound can be significantly improved, providing a strong promoter having great potential for industrial fermentation of target compounds such as amino acids and organic acids.

In some embodiments, the polynucleotide having promoter activity provided by the present disclosure has 0.4 to 13 folds or more improved promoter activity as compared with the promoter activity of the wild-type mdh gene promoter.

In some embodiments, the present disclosure provides a transcription expression cassette, a recombinant expression vector, and a recombinant host cell, which comprise the aforesaid polynucleotide having promoter activity. In the transcription expression cassette, the recombinant expression vector and the recombinant host cell, the polynucleotide having promoter activity is operably ligated with a target gene, and efficient expression of a key gene in the synthesis pathway of a target compound can be realized.

In some embodiments, the present disclosure provides a method for preparing a protein capable of increasing the expression level of a protein related to the synthesis of amino acids, organic acids or gene expression regulatory proteins, thereby enabling efficient production of a target compound.

In some embodiments, the present disclosure provides a method for producing a target compound capable of improving the expression efficiency of a protein related to the synthesis of a target compound by using the aforesaid polynucleotide having promoter activity; and then the yield and conversion rate of the target compound can be effectively improved, and large-scale industrial production of the target compound can be realized.

### BRIEF DESCRIPTION OF THE DRAWINGS

FIG. 1 shows a plasmid profile of pEC-XK99E-P_{mdh}-rfp:

FIG. 2 shows a fluorescence result of mutant clones grown on a culture plate.

### DETAILED DESCRIPTION

When used in combination with the term “comprising” in the claims and/or the specification, the word “a” or “an” may refer to “one”, or refer to “one or more”, “at least one”, and “one or more than one”.

As used in the claims and specification, the term “comprise”, “have/has”, “include”, or “contain” is intended to be inclusive or open-ended, and does not exclude additional or unquoted elements or method steps.

Throughout the present disclosure document, the term “about” means that a value includes the standard deviation of the error of a device or method used to measure the value.

It is applicable to the content disclosed herein that the term “or” is defined only as alternatives and “and/or”, but the term “or” used herein refers to “and/or” unless otherwise expressly stated to be merely alternatives or mutual exclusion between alternatives.

The selected/optional/preferred “numerical range”, when used in the claims or the specification, includes not only the numerical endpoints at both ends of the range, but also all natural numbers covered between the preceding numerical endpoints with respect to these numerical endpoints.

As used herein, the term “malate dehydrogenase” catalyzes the reversible conversion between malic acid and oxaloacetic acid and is coded by mdh gene. In some embodiments, the mdh gene in the present disclosure is derived from Corynebacterium glutamicum.

As used herein, the term “phosphoenolpyruvate carboxylase” (Phosphopyruvate carboxylase) catalyzes the conversion of phosphoenolpyruvate (PEP) to oxaloacetic acid and is coded by ppc gene.

As used herein, the term “pyruvate carboxylase” catalyzes the reversible carboxylation of pyruvic acid to form acetyl oxalate and is coded by pyc gene.

As used herein, the term “polynucleotide” refers to a polymer composed of nucleotides. Polynucleotide can be in the form of an individual fragment or a component of a larger nucleotide sequence structure, and it is derived from a nucleotide sequence separated at least once in quantity or concentration and can be recognized, manipulated and restored from the sequence and its component nucleotide sequences by standard molecular biology methods (such as using a cloning vector). When a nucleotide sequence is represented by a DNA sequence (i.e., A, T, G, C), this also includes an RNA sequence (i.e., A, U, G, C), wherein “U” replaces “T”. In other words, “polynucleotide” refers to a nucleotide polymer that has been removed from other nucleotides (an individual fragment or an entire fragment), or may be a constituent part or component of a larger nucleotide structure, such as an expression vector or a polycistronic sequence. The polynucleotide includes DNA, RNA and cDNA sequences.

As used herein, the term “wild-type” refers to an object that can be found in nature. For example, a polypeptide or a polynucleotide sequence present in an organism that can be isolated from one source in nature and has not been intentionally modified by human in the laboratory is naturally occurring. As used herein, “naturally occurring” and “wild-type” are synonymous. In some embodiments, the wild-type promoter in the present disclosure refers to the wild-type mdh gene promoter, i.e., the polynucleotide having the sequence as shown in SEQ ID NO: 1.

As used herein, the term “mutant” refers to a polynucleotide or polypeptide that, relative to the “wild-type” or “comparative” polynucleotide or polypeptide, comprises alternation(s) (i.e., substitution, insertion and/or deletion of a polynucleotide) at one or more (e.g., several) positions, wherein the substitution refers to a substitution of a different nucleotide for a nucleotide that occupies one position: the deletion refers to removal of a nucleotide that occupies certain position: and the insertion refers to an addition of a nucleotide after the nucleotide adjacent to and immediately following the occupied position.

In some embodiments, the “mutation” in the present disclosure is “substitution”, which is a mutation caused by a substitution of one base in the nucleotide with another different base, which is also referred to as a base substitution or a point mutation.

Specifically; the sequence as set forth in SEQ ID NO: 1 is the sequence of mdh gene promoter, which comprises a promoter core region having a nucleotide sequence of “CGTCAAGATCACCCAAAACTGGTGGCTGTTCTCTTTTAAGCGGGATAGCATGGGTTCTTA”, wherein the underlined part is the sequence of the −10 region. The mutant in the present disclosure introduces mutated nucleotides in the −10 region and the 17 bp before the −10 region, and it is found that the promoter activity of the mutants after the introduction of mutations at the aforesaid positions is significantly enhanced, and a series of novel strong promoters are obtained, providing abundant expression regulatory elements for the efficient synthesis of target compounds.

In some embodiments, the polynucleotide having promoter activity refers to a mutant of the polynucleotide comprising the sequence as set forth in SEQ ID NO: 1, the mutant has mutated nucleotide(s) at one or more positions in position 105 to position 126 of the sequence as set forth in SEQ ID NO: 1 and does not comprise the polynucleotide mutating to TTCTCTTTTAAGCGGGATAGCA at position 105 to position 126 of the sequence as set forth in SEQ ID NO: 1. The mutant has improved promoter activity compared with the polynucleotide comprising the sequence as set forth in SEQ ID NO: 1.

In some embodiments, the mutant of the polynucleotide comprising the sequence as set forth in SEQ ID NO: 1 has 0.4 to 13 folds or more improved promoter activity as compared with the promoter activity of the polynucleotide comprising the sequence as set forth in SEQ ID NO: 1.

In some more specific embodiments, the mutant has 0.4, 1.7, 1.8, 2.0, 2. 1. 2.3. 2.4, 3.1, 3.2. 3.4, 3.6. 4.0, 4.3, 4.5, 4.6, 4.7, 4.8, 4.9, 5.0, 5.1, 5.2, 5.3, 5.6, 5.7, 5.9, 6.1, 6.2, 6.4, 6.5, 6.6, 6.7, 6.8. 7.1. 7.3, 7.4, 7.5, 7.6, 7.7, 7.8, 8.2, 9.6, 13.0 folds improved promoter activity as compared with the promoter activity of the polynucleotide comprising the sequence as set forth in SEQ ID NO: 1.

As used herein, the term “promoter” refers to a nucleic acid molecule, which is typically located upstream of the coding sequence of the target gene, providing a recognition site for RNA polymerase, and is located upstream in the 5′ direction of the transcription initiation site of mRNA. It is an untranslated nucleic acid sequence to which RNA polymerase binds and which initiates transcription of the target gene. In the synthesis of ribonucleic acid (RNA), the promoter can interact with transcription factors and regulate gene transcription and control the initiation time and degree of gene expression (transcription), and it comprises a core promoter region and regulatory region, functions like a “switch”, determines the activity of the gene and then controls which kind of protein the cell starts to produce.

As used herein, the term “promoter core region” refers to a nucleic acid sequence located in the promoter region of prokaryotes, and it is the core sequence region that plays the role of promoter, mainly including the −35 region, the −10 region, and the region between the −35 region and the −10 region, and the transcription initiation site, the −35 region being the recognition site of RNA polymerase, and the −10 region being the binding site of RNA polymerase. In some embodiments, the polynucleotide having promoter activity of the present disclosure is a mutant that comprises a promoter core region of mdh gene and introduces mutation in the −10 region and the 17 bp before the −10 region of the promoter core region, so as to obtain promoter activity significantly improved as compared with the promoter activity of the mdh gene promoter.

As used herein, the term “sequence identity” and “identity percentage” refer to the percentage of nucleotides or amino acids that are the same (i.e., identical) between two or more polynucleotides or polypeptides. The sequence identity between two or more polynucleotides or polypeptides can be determined by aligning the nucleotide or amino acid sequences of polynucleotides or polypeptides, scoring the number of positions at which nucleotide or amino acid residues are identical in the aligned polynucleotides or polypeptides, and comparing the number of these positions with the number of positions at which nucleotide or amino acid residues are different in the aligned polynucleotides or polypeptides. Polynucleotides can be different at one position, for example, by containing a different nucleotide (i.e., substitution or mutation) or deletion of a nucleotide (i.e., nucleotide insertion or nucleotide deletion in one or two polynucleotides). Polypeptides can be different at one position, for example, by containing a different amino acid (i.e., substitution or mutation) or deletion of an amino acid (i.e., amino acid insertion or amino acid deletion in one or two polypeptides). The sequence identity can be calculated by dividing the number of positions at which nucleotide or amino acid residues are identical by the total number of nucleotide or amino acid residues in the polynucleotide or the polypeptide. For example, the identity percentage can be calculated by dividing the number of positions at which nucleotide or amino acid residues are identical by the total number of nucleotide or amino acid residues in the polynucleotide or polypeptide, and multiplying the result by 100.

In some embodiments, two or more sequences or subsequences, when compared and aligned at maximum correspondence by the sequence alignment algorithm or by the visual inspection measurement, have at least 80%, 81%, 82%, 83%, 84%, 85%, 86%, 87%, 88%, 89%, 90%, 91%, 92%, 93%, 94%, 95%, 96%, 97%, 98%, or 99% “sequence identity” or “percent identity” of nucleotides. In some embodiments, the sequence is substantially identical over the full length of either or both of the compared biopolymers (e.g., polynucleotides).

As used herein, the term “complementary” refers to hybridization or base pairing between nucleotides or nucleotides, e.g., between two chains of a double-stranded DNA molecule or between an oligonucleotide primer and a primer binding site on a single-stranded nucleotide to be sequenced or amplified.

As used herein, the term “high-stringent conditions” means that following the standard DNA blotting procedures, a probe of at least 100 nucleotides in length pre-hybridizes or hybridizes for 12 to 24 hours at 42° C. in 5× SSPE (saline sodium phosphate EDTA). 0.3% SDS, 200 μg/ml of cleaved and denatured salmon sperm DNA, and 50% formamide. Finally, the vector material is washed three times at 65° C. with 2× SSC and 0.2% SDS, each for 15 min.

As used herein, the term “very high-stringent conditions” means that following the standard DNA blotting procedures, a probe of at least 100 nucleotides in length pre-hybridizes or hybridizes for 12 to 24 hours at 42° C. in 5× SSPE (saline sodium phosphate EDTA), 0.3% SDS, 200 μg/ml of cleaved and denatured salmon sperm DNA, and 50% formamide. Finally, the vector material is washed three times at 70° C. with 2× SSC and 0.2% SD, each for 15 min.

In some embodiments, the polynucleotide having promoter activity of the present disclosure can be used to initiate the expression of protein-coding genes. In some other embodiments, the polynucleotide having promoter activity of the present disclosure can be used to initiate the expression of non-coding genes.

As used herein, the term “expression” includes any step involving RNA production and protein production, including but not limited to transcription, post-transcriptional modification, translation, post-translational modification and secretion.

As used herein, the term “transcription expression cassette” refers to a recombinant expression element comprising a polynucleotide having promoter activity: In some embodiments, a transcription regulatory element that regulates the target gene may comprise elements such as an enhancer, a silencer, an insulator in addition to the polynucleotide having promoter activity: In some embodiments, the target gene in the present disclosure is specifically a protein-coding gene. “Operably ligating” of a target gene to a polynucleotide having promoter activity means that a polynucleotide having promoter activity is functionally ligated to a target gene to initiate and mediate the transcription of the target gene, and the operably ligating can be in any of the ways described by those skilled in the art.

As used herein, the term “vector” refers to a DNA construct containing a DNA sequence operably ligated with suitable regulatory sequences, so as to express a target gene in a suitable host. “Recombinant expression vector” refers to a DNA structure used to express, for example, a polynucleotide coding a desired polypeptide. The recombinant expression vector may include, for example, i) a collection of genetic elements that have regulatory functions on gene expression, such as promoters and enhancers: ii) a structure or coding sequence transcribed into mRNA and translated into a protein: and iii) transcription subunits which appropriately transcribe and translate the start and stop sequences. The recombinant expression vector is constructed in any suitable way: The property of the vector is not critical, and any vector can be used, including plasmids, viruses, phages and transposons. Possible vectors for use in the present disclosure include, but are not limited to, chromosomal, non-chromosomal and synthetic DNA sequences, such as bacterial plasmids, phage DNA, yeast plasmids and vectors derived from combinations of plasmids and phage DNA, and DNA from viruses such as vaccinia virus, adenovirus, fowl pox virus, baculovirus, SV40 and pseudorabies virus, etc. In the present disclosure, “recombinant expression vector” and “recombinant vector” can be used interchangeably:

As used herein, the term “target RNA” includes functional RNAs that play a role in processes such as genetic coding, translation, regulation, gene expression, etc. In the present disclosure, the target RNA ligated to a polynucleotide having promoter activity can be any functional RNA in the art.

In some embodiments, the target RNA is tRNA or sRNA. In some other embodiments, the target RNA can also be other types of RNA such as sgRNA, crRNA, tracrRNA, miRNA, siRNA, etc.

As used herein, the term “target gene” relates to any of genes that are ligated to the polynucleotide having promoter activity of the present disclosure to regulate its transcriptional level.

In some embodiments, the target gene is a coding gene of a protein related to the synthesis of a target compound. In some embodiments, the target gene is a coding gene of a gene expression regulatory protein. In some embodiments, the target gene is a coding gene of a protein related to membrane transport.

Exemplarily, the target gene is a coding gene that codes an enzyme related to biosynthesis of a target compound, a coding gene of an enzyme related to the reducing power, a coding gene of an enzyme related to glycolysis or TCA cycle, or a coding gene of an enzyme related to the release of a target compound, etc.

Exemplarily, the target gene includes at least one of the following genes: pyruvate carboxylase gene, phosphoenolpyruvate carboxylase gene, γ-glutamyl kinase gene, glutamate semialdehyde dehydrogenase gene, pyrroline-5-carboxylate reductase gene, amino acid transport protein gene, ptsG system related gene, pyruvate dehydrogenase gene, homoserine dehydrogenase gene, oxaloacetic acid decarboxylase gene, gluconic acid repressor gene, glucose dehydrogenase gene, aspartate kinase gene, aspartate semialdehyde dehydrogenase gene, aspartate ammonia lyase gene, dihydrodipicolinate synthase gene, dihydropyridinic acid reductase gene, succinyl diaminopimelate aminotransferase gene, tetrahydrodipicolinate succinylase gene, succinyl diaminopimelate deacylase gene, diaminopimelic acid epimerase gene, diaminopimelic acid deacylase gene, glyceraldehyde-3-phosphate dehydrogenase gene, transketolase gene, diaminopimelate dehydrogenase gene, glyceraldehyde-3-phosphate dehydrogenase gene.

As used herein, the term “target compound” can be selected from amino acids, organic acids, and can also be selected from other types of compounds that can be obtained by biosynthesis in the art.

In some embodiments, the target compound is an “amino acid” or “L-amino acid”.

“Amino acid” or “L-amino acid” typically refers to refers to a basic constituent unit of a protein in which amino and carboxyl group are bounded to the same carbon atom. Exemplarily; the amino acid is selected from one or more of the following: glycine, alanine, valine, leucine, isoleucine, threonine, serine, cysteine, glutamine, methionine, aspartic acid, asparagine, glutamic acid, lysine, arginine, histidine, phenylalanine, tyrosine, tryptophan, proline, hydroxyproline. 5-aminolevulinic acid or derivatives of any of the above amino acids. In addition, the amino acid can also be other types of amino acids in the art.

In some embodiments, the target compound is an organic acid. The organic acid can be an acidic organic compound, e.g., those compounds that contain a carboxyl group and a sulfonic acid group. Exemplarily, the organic acid includes one or more of the following: lactic acid, acetic acid, succinic acid, butyric acid, palmitic acid, oxalic acid, oxaloacetic acid, tartaric acid, citric acid, propionic acid, hexenoic acid, decanoic acid acid, caprylic acid, valeric acid, malic acid or derivatives of any of the above organic acids. In addition, the organic acid can also be any other types of organic acids in the art.

The term “protein-coding gene” in the present disclosure refers to a DNA molecule capable of directing the synthesis of a protein by certain rules, and the process by which a protein-coding gene directing protein synthesis generally includes transcription process using double-stranded DNA as a template and translation using mRNA as a template. The protein-coding gene contains CDS sequence (Coding Sequence) directing the production of protein-coding mRNA.

Exemplarily; the protein-coding gene includes, but is not limited to, coding genes of proteins related to the synthesis of a target compound, and in some embodiments, the protein-coding gene relates to coding genes of proteins related to the synthesis of L-amino acid. Exemplarily: the proteins related to the synthesis of L-amino acid include, but are not limited to, one or a combination of two or more of: pyruvate carboxylase, phosphoenolpyruvate carboxylase, γ-glutamyl kinase, glutamate semialdehyde dehydrogenase, pyrroline-5-carboxylate reductase, amino acid transport protein, ptsG system, pyruvate dehydrogenase, homoserine dehydrogenase, oxaloacetic acid decarboxylase, gluconic acid repressor protein, glucose dehydrogenase. In some embodiments, the protein related to the synthesis of L-amino acid is a protein related to the synthesis of L-lysine, including one or a combination of two or more of: aspartate kinase, aspartate semialdehyde dehydrogenase, aspartate ammonia lyase, dihydrodipicolinate synthase, dihydropyridinic acid reductase, succinyl diaminopimelate aminotransferase, tetrahydrodipicolinate succinylase, succinyl diaminopimelate deacylase, diaminopimelic acid epimerase, diaminopimelic acid deacylase, glyceraldehyde-3-phosphate dehydrogenase, lysine transport protein, transketolase, diaminopimelate dehydrogenase and pyruvate carboxylase.

In some embodiments, the protein-coding gene relates to a coding gene of a protein related to the synthesis of organic acids. Exemplarily; the protein-coding gene is used to code a protein related to the synthesis of oxaloacetic acid, a protein related to the synthesis of citric acid, or is used to code a protein related to the synthesis of succinic acid.

In some embodiments, the protein-coding gene relates to a coding gene of a related enzyme that promotes oxaloacetic acid synthesis. Exemplarily, the protein-coding gene is a coding gene ppc gene of phosphoenolpyruvate carboxylase. As reported in the existing literature^{[1]}, expression of the related enzyme that promotes oxaloacetic acid synthesis can increase the yield of 5-aminolevulinic acid.

In the present disclosure, the term “host cell” refers to any cell type that is easily transformed, transfected, transduced, etc., with a transcription initiation element or expression vector comprising the polynucleotide of the present disclosure. The term “recombinant host cell” covers a host cell that differs from the parent cell after introduction of a transcription initiation element or a recombinant expression vector, and the recombinant host cell is specifically realized by transformation.

In the present disclosure, the term “transformation” has the meaning generally understood by those skilled in the art, i.e., the process of introducing exogenous DNA into a host. The transformation method includes any method for introducing nucleic acid into a cell, including but not limited to electroporation, calcium phosphate (CaPO₄) precipitation, calcium chloride (CaCl₂) precipitation, microinjection, polyethylene glycol (PEG) method, DEAE-dextran method, cationic liposome method and lithium acetate-DMSO method.

The host cell of the present disclosure can be a prokaryotic cell or an eukaryotic cell, as long as it is a cell to which the polynucleotide having promoter activity of the present disclosure can be introduced. In one embodiment, the host cell refers to a prokaryotic cell. Specifically: the host cell is derived from a microorganism suitable for producing amino acids, organic acids by fermentation, such as genus Corynebacterium, genus Brevibacterium, genus Arthrobacterium, genus Microbacterium or genus Escherichia. Preferably, the host cell is Corynebacterium glutamicum from genus Corynebacterium. Among them, Corynebacterium glutamicum can be Corynebacterium glutamicum ATCC 13032, Corynebacterium glutamicum ATCC 13869, or Corynebacterium glutamicum ATCC 14067, and a mutant strain producing amino acids, especially lysine, or a derivative strain of Corynebacterium glutamicum prepared from the above strains. In some embodiments, the host cell in the present disclosure can be any type of strains having the capability for producing amino acids, including wild-type strains and recombinant strains.

Exemplarily; the host cell is a lysine-producing host cell. In some embodiments, the lysine-producing host cell can be a strain that expresses aspartate kinase that relieves feedback inhibition on the basis of Corynebacterium glutamicum ATCC 13032. Besides, the lysine-producing host cell can also be other types of strains that have the capability for producing lysine.

In some embodiments, the lysine-producing host cell may alsoinclude, but is not limited to, one or more genes selected from the following that are attenuated or reduced in expression:
a. adhE gene coding ethanol dehydrogenase;
b. ackA gene coding acetate kinase;
c. pta gene coding phosphate acetyltransferase;
d. ldhA gene coding lactate dehydrogenase;
e. focA gene coding formate transporter;
f. pflB gene coding pyruvate formate lyase;
g. poxB gene coding pyruvate oxidase;
h. thrA gene coding aspartate kinase I/homoserine dehydrogenase I bifunctional enzyme;
i. thrB gene coding homoserine kinase;
j. ldcC gene coding lysine decarboxylase; and
h. cadA gene coding lysine decarboxylase.

In some embodiments, the lysine-producing host cell may also include, but is not limited to, one or more genes selected from the following that are enhanced or overexpressed:
a. dapA gene coding dihydrodipyridine synthase that relieves feedback inhibition of lysine;
b. dapB gene coding dihydrodipicolinate reductase;
c. ddh gene coding diaminopimelate dehydrogenase;
d. dapD coding tetrahydrodipicolinate succinylase and dapE coding succinyl diaminopimelate deacylase
e. asd gene coding aspartate-semialdehyde dehydrogenase;
f. ppc gene coding phosphoenolpyruvate carboxylase;
g. pntAB gene coding nicotinamide adenine dinucleotide transhydrogenase;
i. lysE gene coding lysine transport protein.

Exemplarily; the host cell is a threonine-producing host cell. In some embodiments, the threonine-producing host cell is a strain that expresses aspartate kinase LysC gene that relieves feedback inhibition on the basis of Corynebacterium glutamicum ATCC 13032. In some other embodiments, the threonine-producing host cell can also be other types of strains having threonine-producing capability.

In some embodiments, the threonine-producing host cell contains one or more genes selected from the following that are enhanced or overexpressed:
a. thrABC gene coding threonine operon;
b. hom gene coding homoserine dehydrogenase that relieves feedback inhibition;
c. gap gene coding glyceraldehyde-3-phosphate dehydrogenase;
d. pyc gene coding pyruvate carboxylase;
e. mqo gene coding malate; quinone oxidoreductase;
f. tkt gene coding transketolase;
g. gnd gene coding 6-phosphogluconate dehydrogenase;
h. thrE gene coding threonine exporter;
i. eno gene coding enolase

Exemplarily, the host cell is an isoleucine-producing host cell. In some embodiments, the isoleucine-producing host cell is a strain that produces L-isoleucine by substituting alanine for the amino acid at position 323 of the ilvA gene of L-threonine dehydratase. In some other embodiments, the isoleucine-producing host cell can also be other types of strains having isoleucine-producing capability.

Exemplarily; the host cell is an O-acetylhomoserine-producing host cell. In some embodiments, the O-acetylhomoserine-producing host cell is a strain that produces O-acetylhomoserine by inactivating O-acetylhomoserine (thiol)-lyase. In some other embodiments, the O-acetylhomoserine-producing host cell can also be other types of strains having O-acetylhomoserine production capability.

Exemplarily; the host cell is a methionine-producing host cell. In some embodiments, the methionine-producing host cell is a strain that produces methionine by inactivating transcription regulators of methionine and cysteine. In some other embodiments, the methionine-producing host cell can also be other types of strains having methionine-producing capability.

In the present disclosure, the culture of the host cell can be carried out according to the conventional methods in art, including but not limited to well-plate culture, shaking culture, batch culture, continuous culture and fed-batch culture, etc., and various culture conditions such as temperature, time and pH value of the culture medium can be appropriately adjusted according to the actual situations.

Unless otherwise defined or clearly indicated, all technical and scientific terms in the present disclosure have the same meanings as commonly understood by those of ordinary skill in the art to which the present disclosure pertains.

### Mutant of mdh Gene Promoter Core Region

The present disclosure uses the sequence of the mdh gene promoter core region and introduces mutation in the −10 region and the 17 bp before the −10 region of the mdh gene promoter, to obtain a mutant of mdh gene promoter core region that comprises a mutated −10 region.

For the polynucleotide having promoter activity in the present disclosure, by mutating the mdh gene promoter core region, specifically, by introducing mutations to (TTCTCTTTTAAGCGGGATAGCA) in the vicinity of the −10 region of the mdh gene promoter core region, the mutants in the present disclosure have significantly improved promoter activity as compared with the wild-type promoter that comprises a mdh gene promoter core region, and are a series of novel strong promoters: the mutants, when applied to the fermentation of a target compound, exhibit higher conversion rate and yield of the target compound as compared with the wild-type promoter.

In some embodiments, the polynucleotide having promoter activity in the present disclosure has mutated nucleotides at 1, 2, 3, 4, 5, 6, 7, 8, 9, 10, 11, 12, 13, 14, 15, 16, 17, 18, 19 or 20 positions in position 105 to position 126 of the sequence as set forth in SEQ ID NO: 1, and has improved promoter activity as compared with the wild-type mdh gene promoter having the sequence as set forth in SEQ ID NO: 1.

In some embodiments, the polynucleotide having promoter activity in the present disclosure further includes a polynucleotide that is reversely complementary to the nucleotide sequence of the mutant of the mdh gene promoter as set forth in SEQ ID NO: 1. Moreover, the polynucleotide has improved promoter activity as compared with the wild-type mdh gene promoter having the sequence as set forth in SEQ ID NO: 1.

In some embodiments, the polynucleotide having promoter activity in the present disclosure further includes a polynucleotide that is reversely complementary to the sequence that hybridizes with the nucleotide sequence of a mutant of the mdh gene promoter as set forth in SEQ ID NO: 1 under high stringent hybridization conditions or very high stringent hybridization conditions. Moreover, the polynucleotide has a nucleotide sequence that is not TTCTCTTTTAAGCGGGATAGCA at position 105 to position 126 of the sequence as set forth in SEQ ID NO: 1: and the polynucleotide has improved promoter activity as compared with the wild-type mdh gene promoter having the sequence as set forth in SEQ ID NO: 1.

In some embodiments, the polynucleotide having promoter activity in the present disclosure has at least 80%, 81%, 82%, 83%, 84%, 85%, 86%, 87%, 88%, 89%, 90%, 91%, 92%, 93%, 94%, 95%, 96%, 97%, 98%, 99% sequence identity (including all ranges and percentages between these numbers) compared with the aforesaid polynucleotide. Moreover, the polynucleotide has a nucleotide sequence that is not TTCTCTTTTAAGCGGGATAGCA at position 105 to position 126 of the sequence as set forth in SEQ ID NO: 1: and the polynucleotide has improved promoter activity as compared with the wild-type mdh gene promoter having the sequence as set forth in SEQ ID NO: 1.

In some specific embodiments, the nucleotide sequence of the mutant corresponding to position 105 to position 126 of the sequence as set forth in SEQ ID NO: 1 is selected from any one of the group consisting of the following (P_{mdh-1}) to (P_{mdh-49}):

(P_{mdh}-1)
CAGGTAACAACTGCGAGTACGG;
(P_{mdh}-2)
AGTCTGAGGGATATAATTAAGA;
(P_{mdh}-3)
GGTCGTGGAGGACAAGTTAAGA;
(P_{mdh}-4)
CTACTACCATCGGGTGCTAAGG;
(P_{mdh}-5)
GATAGGTAGGCGTTAAGTAAAC;
(P_{mdh}-6)
GGCAATTCGCGGGGAGGTAGAC;
(P_{mdh}-7)
TATTGGGCGCGGCATTATAGGA;
(P_{mdh}-8)
GGGAACTGCTTTGGTAGTATCA;
(P_{mdh}-9)
GTTCAGGTTTGGGCCGCTATGC;
(P_{mdh}-10)
TGATCGGGGCGCTTTGGTACGG;
(P_{mdh}-11)
TATGAAATAAATTGGAGTAGGC;
(P_{mdh}-12)
AACGGTATCTTTCTCCATATAA;
(P_{mdh}-13)
AACGGAGCGTGTACGACTAGAA;
(P_{mdh}-14)
TGCGCGGGTAAACGCTGTAGAG;
(P_{mdh}-15)
TTGATGTCGTAGCGTAGTATTA;
(P_{mdh}-16)
AGCTCGGACTTTGGTGGTAGGC;
(P_{mdh}-17)
CCGGCGCATTATAACGCTACAA;
(P_{mdh}-18)
CGGGGGTGTAAGTCGAGTAGAC;
(P_{mdh}-19)
ACGGGTAGGTATTTCGCTAGGA;
(P_{mdh}-20)
TAGTGTATGCTTTTACATAGAC;
(P_{mdh}-21)
AGGCAGCATGTACATGATATCG;
(P_{mdh}-22)
TTGAGCGGAGTTAATAGTACAC;
(P_{mdh}-23)
ATACACACAACTTTCGATACTA;
(P_{mdh}-24)
ATTCCAACCTTATGTTATACTG;
(P_{mdh}-25)
GAGGTAGTCGTTTGGGGTAGGA;
(P_{mdh}-26)
ATTGCTCGCGTAAATGGTAAAC;
(P_{mdh}-27)
TGTTATTATGGGAGATGTAAGT;
(P_{mdh}-28)
GAAAAATGTACGTGTGTTAAAA;
(P_{mdh}-29)
AGTCCTCAGTGGAGTGGTAACA;
(P_{mdh}-30)
TTTCCGGTGTTTTCGCGTAGAG;
(P_{mdh}-31)
GGCGTCGGTAGGTATGTTATTG;
(P_{mdh}-32)
ACGGCGATCAGAGTGTGTAAGA;
(P_{mdh}-33)
CCGTGCGTGATATGGGGTAGTG;
(P_{mdh}-34)
GGGTTGTGAGTTATCGGTATAA;
(P_{mdh}-35)
TGCTCCGATGATGGTGGTAAGA;
(P_{mdh}-36)
GTTCTCACGGGTCCGTGTATGA;
(P_{mdh}-37)
TTTCGGAATGGATGGTGTACGC;
(P_{mdh}-38)
ATTATGTCTTCTTTTGGTAAAA;
(P_{mdh}-39)
GATCATAGGTTCTGTGGTAGAT;
(P_{mdh}-40)
CATGGCCGATCATGATGTAATA;
(P_{mdh}-41)
TAGTGGCGGTGTACGTGTATGA;
(P_{mdh}-42)
ATTTGTTACAGGTATGGTAGTT;
(P_{mdh}-43)
ACGGAACTTATTTCTGGTAACG;
(P_{mdh}-44)
ACCGGGAGGACTAATGGTATGG;
(P_{mdh}-45)
GCCACCCCTTTTTCTGCTATTA;
(P_{mdh}-46)
TTGCGCCGGAGTTGCGGTATAG;
(P_{mdh}-47)
GTTTACGGGTGTTCATGTAGTA;
(P_{mdh}-48)
TTTGTCTCGTATCTGTGTAAGA;
(P_{mdh}-49)
ATTTTGGGCACTTAGTGTATTG.

In some specific embodiments, the nucleotide sequence of the mutant is selected from the sequence as set forth in any one of SEQ ID NOs: 3 to 51.

In some embodiments, the polynucleotide having promoter activity in the present disclosure has 0.4 to 13 folds or more improved promoter activity as compared with the promoter activity of the polynucleotide having the sequence as set forth in SEQ ID NO: 1, and further has 0.4, 1.7, 1.8, 2.0, 2.1, 2.3, 2.4, 3.1, 3.2, 3.4, 3.6, 4.0, 4.3, 4.5, 4.6, 4.7, 4.8, 4.9, 5.0, 5.1, 5.2, 5.3, 5.6, 5.7, 5.9, 6.1, 6.2, 6.4, 6.5, 6.6, 6.7, 6.8, 7.1, 7.3, 7.4, 7.5, 7.6, 7.7, 7.8, 8.2, 9.6, 13.0 folds enhanced promoter activity as compared with the promoter activity of a polynucleotide comprising the sequence as set forth in SEQ ID NO: 1.

### Recombinant Expression Vector and Recombinant Host Cell

In some embodiments, amplification is carried out in the present disclosure using ATCC13032 genome (Corynebacterium glutamicum ATCC 13032, Gene ID: 2830649) as a template, and mdh-1 and mdh-2 as primers to obtain a DNA fragment of the mdh gene promoter; the N-terminus 180-bp fragment is amplified using gapN180-1 and gapN180-2 as primers, and a synthetic N-terminus 180 bp of gapN gene derived from Streptococcus mutans 25 as a template: pEC-XK99E plasmid skeleton is amplified using PEC-XK99E-rfp plasmid as a template, and pEC-1 and pEC-2 as primers: DNA fragments of red fluorescent protein gene comprising a linker peptide are amplified using RFP-1/2 as primers, and PEC-XK99E-rfp plasmid as a template. The aforesaid mdh gene promoter fragment, N-terminus 180 bp fragment of gapN. DNA fragments of red fluorescent protein gene comprising a linker peptide, and pEC-XK99E plasmid skeleton are recombinantly ligated, to obtain a recombinant expression vector pEC-XK99E-P_{mdh}-rfp.

In some embodiments, using pEC-XK99E-P_{mdh}-rfp as a template, a fragment comprising a mutation region is amplified using mdh-NI primer and TK-1 primer, a plasmid skeleton fragment is amplified using mdh-N2 primer and TK-2 primer in the present disclosure, and the aforesaid two kinds of fragments are recombinantly ligated to obtain a library of mdh gene promoter mutants.

In some embodiments, Corynebacterium glutamicum ATCC13032 is transformed using a plasmid of the library of mdh gene promoter mutants and pEC-XK99E-P_{mdh}-rfp, respectively, to obtain a recombinant host cell in the present disclosure. Mutants having enhanced fluorescence intensity are screened after well-plate culture, to obtain mdh gene promoter mutants having improved promoter activity:

In some embodiments, amplification is carried out using a plasmid of the library of mdh gene promoter mutants as a template, and mdh-P1, mdh-P2 as primers to obtain fragments of the respective mdh gene promoter mutants in the present disclosure: ppc gene fragments are amplified using Corynebacterium glutamicum ATCC 13032 genome as a template, and ppc-1/ppc-2 as primers; a plasmid skeleton is amplified using pEC-XK99E plasmid as a template, and PEC-1/PEC-2 as primers. The respective promoter mutant fragments are recombinantly ligated to the ppc gene fragments and the plasmid skeleton, to obtain mdh gene promoter mutant plasmids.

In some specific embodiments, the mdh gene promoter mutant plasmids include any one of the following: pEC-P_{mdh-1}-ppc, pEC-P_{mdh-2}-ppc, PEC-P_{mdh-3}-ppc, pEC-P_{mdh-4}-ppc, pEC-P_{mdh-5}-ppc, pEC-P_{mdh-6}-ppc, pEC-P_{mdh-7}-ppc, pEC-P_{mdh-8}-ppc, pEC-P_{mdh-9}-ppc, pEC-P_{mdh-10}-ppc, pEC-P_{mdh-11}-ppc, pEC-P_{mdh-12}-ppc, pEC-P_{mdh-13}-ppc, pEC-P_{mdh-14}-ppc, pEC-P_{mdh-15}-ppc, pEC-P_{mdh-16}-ppc, pEC-P_{mdh-17}-ppc, pEC-P_{mdh-18}-ppc, pEC-P_{mdh-19}-ppc, pEC-P_{mdh-20}-ppc, pEC-P_{mdh-21}-ppc, pEC-P_{mdh-22}-ppc, PEC-P_{mdh-23}-ppc, pEC-P_{mdh-24}-ppc, pEC-P_{mdh-25}-ppc, pEC-P_{mdh-26}-ppc, pEC-P_{mdh-27}-ppc, pEC-P_{mdh-28}-ppc, pEC-P_{mdh-29}-ppc, pEC-P_{mdh-30}-ppc, pEC-P_{mdh-31}-ppc, pEC-P_{mdh-32}-ppc, pEC-P_{mdh-33}-ppc, pEC-P_{mdh-34}-ppc, pEC-P_{mdh-35}-ppc, pEC-P_{mdh-36}-ppc, pEC-P_{mdh-37}-ppc, pEC-P_{mdh-38}-ppc, pEC-P_{mdh-39}-ppc, pEC-P_{mdh-40}-ppc, pEC-P_{mdh-41}-ppc, pEC-P_{mdh-42}-ppc, pEC-P_{mdh-43}-ppc, pEC-P_{mdh-44}-ppc, pEC-P_{mdh-45}-ppc, pEC-P_{mdh-46}-ppc, pEC-P_{mdh-47}-ppc, pEC-P_{mdh-48}-ppc, pEC-P_{mdh-49}-ppc.

In some other embodiments, a required recombinant vector can also be constructed in the present disclosure using the promoter mutant as set forth in any one of (P_{mdh-1}) to (P_{mdh-49}), according to the specific cloning needs.

In some embodiments, a Corynebacterium glutamicum SCgL30 strain in the present disclosure, the threonine at position 311 of aspartate kinase (coded by lysC gene) on the Corynebacterium glutamicum ATCC 13032 genome is mutated into isoleucine, to construct a strain SCgL30 that has certain lysine synthesis capability.

In some specific embodiments, a recombinant expression vector as set forth in any one of pEC-P_{mdh-1}-ppc to pEC-P_{mdh-49}-ppc is transformed into a SCgL30 strain to obtain a recombinant host cell in the present disclosure, and exemplarily; a recombinant expression vector as set forth in pEC-P_{mdh-39}-ppc is transformed into a SCgL30 strain to obtain a recombinant host cell in the present disclosure. In some other specific embodiments, a recombinant vector comprising the promoter mutant as set forth in any one of (P_{mdh-1}) to (P_{mdh-49}) can also be transformed into a SCgL30 strain to obtain a recombinant host cell in the present disclosure.

### Production Process of a Target Compound

(1) A polynucleotide having promoter activity is operably ligated to a coding gene of a protein related to the synthesis of the target compound or to a coding gene of a gene expression regulatory protein, to obtain a recombinant expression vector of the protein related to the synthesis of the target compound or the gene expression regulatory protein, and the recombinant expression vector is used to transform a host cell, to obtain a recombinant host cell.
(2) The recombinant host cell is subject to fermentation culture, and the target compound is collected from the recombinant host cell or the culture broth of the recombinant host cell to complete the production process of the target compound.

In the aforesaid production process, since the polynucleotide has improved promoter activity; in the recombinant host cell, the transcription activity of the coding gene of the protein related to the synthesis of the target compound or the gene expression regulatory protein is increased, and the expression level of the protein related to the synthesis of the target compound or the gene expression regulatory protein is increased, thereby the yield of target compound is significantly increased.

In some embodiments, the target compound is an amino acid, and the coding gene of a protein related to the synthesis of the target compound refers to a coding gene of a protein related to the synthesis of an amino acid. In some embodiments, the target compound is L-amino acid, and the coding gene of a protein related to the synthesis of an amino acid refers to a coding gene of a protein related to the synthesis of L-amino acid.

In some specific embodiments, the protein related to the synthesis of an amino acid is phosphoenolpyruvate carboxylase. Increasing the expression of ppc by using a polynucleotide having promoter activity can enhance the synthesis from phosphoenolpyruvate (PEP) to oxaloacetic acid, which in turn promotes the production of an oxaloacetic acid precursor supply-dependent target compound, including aspartic acid family amino acids (lysine, threonine, isoleucine, methionine), and glutamic acid family amino acids (glutamic acid, proline, hydroxyproline, arginine, glutamine), etc.

In some specific embodiments, the host cell is Corynebacterium glutamicum, an important strain for producing target compounds such as amino acids, organic acids, etc., and after modification of Corynebacterium glutamicum using a polynucleotide, a transcription expression cassette or a recombinant expression vector having a strong constitutive promoter activity, the expression level of the protein related to the synthesis of the target compound in Corynebacterium glutamicum is significantly increased, resulting in a much higher capacity for long-term fermentative accumulation of the target compound in Corynebacterium glutamicum.

In some specific embodiments, the host cell is Corynebacterium glutamicum modified as follows: threonine at position 311 of aspartate kinase (coded by lys(' gene) on the Corynebacterium glutamicum ATCC 13032 genome is mutated into isoleucine.

In some specific embodiments, the culture conditions of recombinant host cells are as follows: the recombinant host cells are inoculated into TSB liquid medium and cultured, and the culture is inoculated as a seed into a 24-well plate of which each well contains fermentation medium, and cultured at 30° C. for 18 hours at a rotation speed of the plate shaker of 800 rpm. L-lysine production yield is detected after the fermentation.

Ingredients of the fermentation medium for lysine are as follows: glucose, 80 g/L: yeast powder, 1 g/L: soybean peptone, 1 g/L: NaCl, 1 g/L: Ammonium sulfate, 1 g/L: Urea, 10 g/L: K₂HPO₄·3H₂O, 1 g/L: MgSO₄·7H₂O, 0.45 g/L: FeSO₄·7H₂O, 0.05 g/L: biotin, 0.4 mg/L: vitamin B1, 0.1 mg/L: MOPS, 40 g/L: initial pH 7.2. 25 μg/mL Kanamycin is supplemented to the medium.

In some specific embodiments, target compounds can be recovered from recombinant host cells or culture broths of recombinant cells by methods commonly used in the art, including but not limited to filtration, anion exchange chromatography; crystallization and HPLC.

In the art, methods for manipulating microorganisms are known, as explained in publications such as Current Protocols in Molecular Biology (Online ISBN: 9780471142720, John Wiley and Sons, Inc.), Microbial Metabolic Engineering: Methods and Protocols (Qiong Cheng Ed., Springer) and Systems Metabolic Engineering: Methods and Protocols (Hal S. Alper Ed., Springer).

### EXAMPLES

Other objects, features and advantages of the present disclosure will become apparent from the following detailed description. However, it should be understood that the detailed description and specific examples (although indicating specific embodiments of the present disclosure) are given for explanatory purpose only; because various variations and modifications within the spirit and scope of the present disclosure will become obvious to those skilled in the art upon reading the detailed description.

The experimental techniques and methods used in the examples are conventional technical methods unless otherwise specified. For example. the experimental methods for which specific conditions are not indicated in the following examples are usually in accordance with conventional conditions, such as those described in Molecular Cloning: Laboratory Manual (New York: Cold Spring Harbor Laboratory Press. 1989), by Sambrook et al., or those suggested by manufacturers. Unless otherwise specified, the materials and reagents used in the examples can be obtained through formal commercial channels.

### Example 1. Construction of Intensity Characterization Plasmid of Corynebacterium glutamicum mdh Gene Promoter

In the present disclosure, the promoter of Corynebacterium glutamicum malate dehydrogenase mdh (malate dehydrogenase) gene was selected to carry out intensity characterization, and a specific region mutation was further introduced to construct a promoter library: to obtain promoter mutants having enhanced expression intensity: In bacteria, the expression regulatory sequence and the N-terminus coding region of a gene are the key regions that affect gene expression. In the present disclosure, the expression intensity of a target promoter was characterized based on fluorescence intensity: using a method of sequentially ligating a gene upstream promoter, the N-terminus 180 bp coding region of a Streptococcus mutans-derived NADP⁺-dependent glyceraldehyde-3-phosphate dehydrogenase gapN gene, a flexible linker peptide and a red fluorescent protein gene rfp.

The present example first constructed a characterization vector of the Corynebacterium glutamicum mdh gene promoter. Based on the skeleton of a pEC-XK99E plasmid, 60) amino acids at the N-terminus of Streptococcus mutans gapN gene, a linker peptide and a red fluorescent protein gene were expressed by an expression regulatory region comprising a promoter upstream of mdh gene. The specific construction was as follows:

### (1) Amplification of Fragments of mdh Gene Promoter and N-terminus Sequence

Amplification primers were designed according to the published genome sequence of Corynebacterium glutamicum ATCC13032 (Gene ID:2830649), the annotation information of Corynebacterium glutamicum mdh gene, and the synthetic N-terminus 180 bp of Streptococcus mutans derived gapN gene.

A mdh wild-type promoter (the sequence is as set forth in SEQ ID NO: 1) was amplified using mdh-1/2 as primers and ATCC13032 genome as the template: the N-terminus 180 bp fragment was amplified using gapN180-1/2 as primers and the synthetic N-terminus 180 bp of Streptococcus mutans derived gapN gene (the sequence is as set forth in SEQ ID NO: 2) as a template.

### (2) Amplification of Plasmid Skeleton and rfp Fragment

By using pEC-XK99E-rfp^{[}2] plasmid reported in literatures as a template, pEC-XK99E plasmid skeleton was amplified using pEC-1/2 as primers, and the DNA fragment of the red fluorescent protein gene comprising a linker peptide (the DNA sequence is GGCGGTGGCTCTGGAGGTGGTGGGTCCGGCGGTGGCTCT) were amplified using RFP-1/2 as primers.

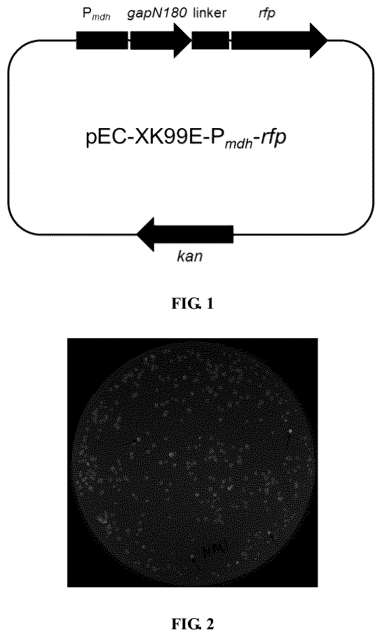

The above-obtained mdh gene promoter fragment, the N-terminus 180 bp fragment of gapN, the DNA fragment of red fluorescent protein gene comprising a linker peptide, and the plasmid skeleton were cloned and ligated via Vazyme's One Step Cloning Kit to obtain a pEC-XK99E-P_{mdh}-rfp characterization vector, of which the plasmid map is shown in FIG. 1. The sequences of the primers used above are shown in Table 1.

TABLE 1

| Primers | Nucleotide sequence | SEQ ID NO. |
|---|---|---|
| mdh-1 | CCTGATGCGGTATTTTCTCCCG | SEQ ID NO: 52 |
| | ATTCCCCATGATGCCCTAAC | |
| mdh-2 | TAAACAATCCTCAATCCTTGTAGG | SEQ ID NO: 53 |
| gapN180-1 | CAAGGATTGAGGATTGTTTAATG | SEQ ID NO: 54 |
| | ACAAAACAATATAAAAATTATGTC | |
| gapN180-2 | CCACCTCCAGAGCCACCGCCTGAA | SEQ ID NO: 55 |
| | AGTGATCGCCAAGCTGG | |
| pEC-1 | CTGCAGGCATGCAAGCTTGG | SEQ ID NO: 56 |
| pEC-2 | GGAGAAAATACCGCATCAGGC | SEQ ID NO: 57 |
| RFP-1 | GGCGGTGGCTCTGGAGGTGGTGG | SEQ ID NO: 58 |
| | GTCCGGCGGTGGCTCTGCTTCCT | |
| | CCGAAGACGTTATCAAAG | |
| RFP-2 | CCAAGCTTGCATGCCTGCAGTTA | SEQ ID NO: 59 |
| | AGCACCGGTGGAGTGACGAC | |

### Example 2 Screening and Intensity Characterization of Mutants of Corynebacterium glutamicum mdh Gene Promoter

(1) Construction of mutant library of Corynebacterium glutamicum mdh gene promoter and preliminary screening

In the present disclosure, a series of mutations were introduced into the −10 region and the upstream 17 bp sequence of the Corynebacterium glutamicum mdh gene promoter, to construct a promoter library. The mdh promoter core region is as follows, wherein the underlined part is the main sequence of the −10 region of the promoter:

CGTCAAGATCACCCAAAACTGGTGGCTGTTCT
CTTTTAAGCGGGATAGCATGGGTTCTTA;

The sequence in which mutations were introduced is as follows:

CGTCAAGATCACCCAAAACTGGTGGCTGNNN
NNNNNNNNNNNNNNTANNNTGGGTTCTTA.

By using the pEC-XK99E-P_{mdh}-rfp characterization vector constructed in Example 1 as a template, a fragment comprising the mutation region was amplified using mdh-NI primer and TK-1 primer, and the plasmid skeleton fragment was amplified using mdh-N2 primer and TK-2 primer. The above two fragments were ligated via Vazyme's One Step Cloning Kit, and all clones obtained by gene promoter mutation were collected and plasmids were extracted to obtain a plasmid library of mdh gene promoter mutants.

Corynebacterium glutamicum ATCC13032 was transformed with the above plasmid library, and coated on a TSB solid plate supplemented with 25 μg/mL kanamycin. The plate, on which hundreds of clones were grown, was fluorescence photographed with a fluorescence imaging system, and mutants with improved expression intensity were preliminarily screened according to the fluorescence brightness of the clones. Ingredients (g/L) of the TSB plate medium are as follows: glucose, 5 g/L: yeast powder, 5 g/L; soy peptone, 9 g/L: Urea, 3 g/L; succinic acid, 0.5 g/L; K₂HPO₄·3H₂O, 1 g/L: MgSO₄·7H₂O, 0.1 g/L; biotin, 0.01 mg/L: vitamin B1, 0.1 mg/L: MOPS, 20 g/L: agar powder, 15 g/L. Meanwhile, Corynebacterium glutamicum ATCC13032 was transformed with the wild-type promoter characterization plasmid obtained in Example 1 to obtain a control strain. In the present disclosure, more than 10,000 clones of the promoter library were preliminarily screened to obtain mutants with enhanced fluorescence brightness. The screening plate is as shown in FIG. 2. The sequences of the primers used above are shown in Table 2.

TABLE 2

| Primers | Nucleotide sequence | SEQ ID NO. |
|---|---|---|
| mdh-N1 | TCACCCAAAACTGGTGGCTGNNN | SEQ ID NO: 60 |
| | NNNNNNNNNNNNNNTANNNTGG | |
| | GTTCTTAGAGGACCCCCTAC | |
| mdh-N2 | CAGCCACCAGTTTTGGGTGATC | SEQ ID NO: 61 |
| TK-1 | AACCTTCCATACGAACTTTGAA | SEQ ID NO: 62 |
| | ACG | |
| TK-2 | CAAAGTTCGTATGGAAGGTTCCG | SEQ ID NO: 63 |

(2) Characterization and Sequence Analysis of the Library of Corynebacterium glutamicum Gene Promoter Mutants

All mutants with enhanced fluorescence brightness shown by the fluorescence imaging in the above plate were cultured in a 96-well plate to characterize the intensity of promoters. Ingredients (g/L) of the TSB liquid medium are as follows: glucose, 5 g/L: yeast powder, 5 g/L: soy peptone, 9 g/L: Urea, 3 g/L: succinic acid, 0.5 g/L: K₂HPO₄·3H₂O, 1 g/L; MgSO₄·7H₂O, 0.1 g/L; biotin, 0.01 mg/L: vitamin B1, 0.1 mg/L; MOPS, 20 g/L. 25 μg/mL Kanamycin was added to the culture medium. The clones with enhanced fluorescence brightness obtained in the plate and the wild-type promoter control strain were inoculated with toothpicks into a 96-well plate containing 200 μl TSB liquid culture medium in each well, with three parallel strains for each strain. At a rotation speed of the plate shaker of 800 rpm, after culturing at 30° C. for 24 hours, the fluorescence intensities of the strains were detected. For the strains with improved fluorescence intensity as compared with the wild-type control, the mutation region fragments were amplified using pEC-F(TACGGTTCCTGGCCTTTTGC) and RFP-CX (CGGGTGTTTAACGTAAGCTTTG) primers and sequenced.

The characterization and sequencing results of the mdh promoter mutants are shown in Table 3. In the mdh gene promoter library, 49 promoter mutants (the nucleotide sequences of the corresponding mutant promoters were SEQ ID NOs: 3 to 51) with enhanced expression intensity as compared with the wild-type promoter were successfully obtained, and the improved folds ranged from 0.4 to 13.0 folds, indicating that mutations in the −10 region and the vicinity sequences of the mdh gene promoter could significantly enhance the promoter activity and provide plentiful elements for enhancing the expression of target genes.

TABLE 3

| | Fluorescence | Multiple | Sequence of promoter | Sequence No. of |
|---|---|---|---|---|
| | intensity | over | mutation region | the complete |
| Promoter | (RFU/ | the wild- | (positions 105 to 127 | promoter |
| No. | OD600) | type | of SEQ ID NO: 1) | sequence |
| P_{mdh}-WT | 1662 ± 54 | — | TTCTCTTTTAAGCGGGATAGCAT | SEQ ID NO: 1 |
| P_{mdh}-1 | 2362 ± 83 | 0.4 | CAGGTAACAACTGCGAGTACGGT | SEQ ID NO: 3 |
| P_{mdh}-2 | 4469 ± 170 | 1.7 | AGTCTGAGGGATATAATTAAGAT | SEQ ID NO: 4 |
| P_{mdh}-3 | 4543 ± 84 | 1.7 | GGTCGTGGAGGACAAGTTAAGAT | SEQ ID NO: 5 |
| P_{mdh}-4 | 4734 ± 511 | 1.8 | CTACTACCATCGGGTGCTAAGGT | SEQ ID NO: 6 |
| P_{mdh}-5 | 5023 ± 60 | 2.0 | GATAGGTAGGCGTTAAGTAAACT | SEQ ID NO: 7 |
| P_{mdh}-6 | 5118 ± 89 | 2.1 | GGCAATTCGCGGGGAGGTAGACT | SEQ ID NO: 8 |
| P_{mdh}-7 | 5491 ± 262 | 2.3 | TATTGGGCGCGGCATTATAGGAT | SEQ ID NO: 9 |
| P_{mdh}-8 | 5617 ± 117 | 2.4 | GGGAACTGCTTTGGTAGTATCAT | SEQ ID NO: 10 |
| P_{mdh}-9 | 6770 ± 113 | 3.1 | GTTCAGGTTTGGGCCGCTATGCT | SEQ ID NO: 11 |
| P_{mdh}-10 | 6954 ± 503 | 3.2 | TGATCGGGGCGCTTTGGTACGGT | SEQ ID NO: 12 |
| P_{mdh}-11 | 7319 ± 213 | 3.4 | TATGAAATAAATTGGAGTAGGCT | SEQ ID NO: 13 |
| P_{mdh}-12 | 7616 ± 194 | 3.6 | AACGGTATCTTTCTCCATATAAT | SEQ ID NO: 14 |
| P_{mdh}-13 | 8237 ± 162 | 4.0 | AACGGAGCGTGTACGACTAGAAT | SEQ ID NO: 15 |
| P_{mdh}-14 | 8837 ± 136 | 4.3 | TGCGCGGGTAAACGCTGTAGAGT | SEQ ID NO: 16 |
| P_{mdh}-15 | 9131 ± 744 | 4.5 | TTGATGTCGTAGCGTAGTATTAT | SEQ ID NO: 17 |
| P_{mdh}-16 | 9288 ± 128 | 4.6 | AGCTCGGACTTTGGTGGTAGGCT | SEQ ID NO: 18 |
| P_{mdh}-17 | 9393 ± 119 | 4.7 | CCGGCGCATTATAACGCTACAAT | SEQ ID NO: 19 |
| P_{mdh}-18 | 9587 ± 429 | 4.8 | CGGGGGTGTAAGTCGAGTAGACT | SEQ ID NO: 20 |
| P_{mdh}-19 | 9779 ± 472 | 4.9 | ACGGGTAGGTATTTCGCTAGGAT | SEQ ID NO: 21 |
| P_{mdh}-20 | 10045 ± 254 | 5.0 | TAGTGTATGCTTTTACATAGACT | SEQ ID NO: 22 |
| P_{mdh}-21 | 10137 ± 194 | 5.1 | AGGCAGCATGTACATGATATCGT | SEQ ID NO: 23 |
| P_{mdh}-22 | 10266 ± 263 | 5.2 | TTGAGCGGAGTTAATAGTACACT | SEQ ID NO: 24 |
| P_{mdh}-23 | 10341 ± 63 | 5.2 | ATACACACAACTTTCGATACTAT | SEQ ID NO: 25 |
| P_{mdh}-24 | 10547 ± 148 | 5.3 | ATTCCAACCTTATGTTATACTGT | SEQ ID NO: 26 |
| P_{mdh}-25 | 11031 ± 608 | 5.6 | GAGGTAGTCGTTTGGGGTAGGAT | SEQ ID NO: 27 |
| P_{mdh}-26 | 11147 ± 222 | 5.7 | ATTGCTCGCGTAAATGGTAAACT | SEQ ID NO: 28 |
| P_{mdh}-27 | 11440 ± 452 | 5.9 | TGTTATTATGGGAGATGTAAGTT | SEQ ID NO: 29 |
| P_{mdh}-28 | 11464 ± 901 | 5.9 | GAAAAATGTACGTGTGTTAAAAT | SEQ ID NO: 30 |
| P_{mdh}-29 | 11744 ± 386 | 6.1 | AGTCCTCAGTGGAGTGGTAACAT | SEQ ID NO: 31 |
| P_{mdh}-30 | 11894 ± 528 | 6.2 | TTTCCGGTGTTTTCGCGTAGAGT | SEQ ID NO: 32 |
| P_{mdh}-31 | 11948 ± 287 | 6.2 | GGCGTCGGTAGGTATGTTATTGT | SEQ ID NO: 33 |
| P_{mdh}-32 | 12260 ± 494 | 6.4 | ACGGCGATCAGAGTGTGTAAGAT | SEQ ID NO: 34 |
| P_{mdh}-33 | 12440 ± 113 | 6.5 | CCGTGCGTGATATGGGGTAGTGT | SEQ ID NO: 35 |
| P_{mdh}-34 | 12582 ± 872 | 6.6 | GGGTTGTGAGTTATCGGTATAAT | SEQ ID NO: 36 |
| P_{mdh}-35 | 12802 ± 244 | 6.7 | TGCTCCGATGATGGTGGTAAGAT | SEQ ID NO: 37 |
| P_{mdh}-36 | 12833 ± 384 | 6.7 | GTTCTCACGGGTCCGTGTATGAT | SEQ ID NO: 38 |
| P_{mdh}-37 | 12867 ± 750 | 6.7 | TTTCGGAATGGATGGTGTACGCT | SEQ ID NO: 39 |
| P_{mdh}-38 | 13044 ± 666 | 6.8 | ATTATGTCTTCTTTTGGTAAAAT | SEQ ID NO: 40 |
| P_{mdh}-39 | 13420 ± 476 | 7.1 | GATCATAGGTTCTGTGGTAGATT | SEQ ID NO: 41 |
| P_{mdh}-40 | 13792 ± 318 | 7.3 | CATGGCCGATCATGATGTAATAT | SEQ ID NO: 42 |
| P_{mdh}-41 | 13879 ± 271 | 7.4 | TAGTGGCGGTGTACGTGTATGAT | SEQ ID NO: 43 |
| P_{mdh}-42 | 14143 ± 452 | 7.5 | ATTTGTTACAGGTATGGTAGTTT | SEQ ID NO: 44 |
| P_{mdh}-43 | 14191 ± 440 | 7.5 | ACGGAACTTATTTCTGGTAACGT | SEQ ID NO: 45 |
| P_{mdh}-44 | 14352 ± 330 | 7.6 | ACCGGGAGGACTAATGGTATGGT | SEQ ID NO: 46 |
| P_{mdh}-45 | 14490 ± 574 | 7.7 | GCCACCCCTTTTTCTGCTATTAT | SEQ ID NO: 47 |
| P_{mdh}-46 | 14704 ± 537 | 7.8 | TTGCGCCGGAGTTGCGGTATAGT | SEQ ID NO: 48 |
| P_{mdh}-47 | 15265 ± 125 | 8.2 | GTTTACGGGTGTTCATGTAGTAT | SEQ ID NO: 49 |
| P_{mdh}-48 | 17560 ± 1126 | 9.6 | TTTGTCTCGTATCTGTGTAAGAT | SEQ ID NO: 50 |
| P_{mdh}-49 | 23336 ± 967 | 13.0 | ATTTTGGGCACTTAGTGTATTGT | SEQ ID NO: 51 |

### Example 3 Application of Corynebacterium glutamicum mdh Gene Promoter Mutants in L-Lysine Production

(1) Construction of Strains of Corynebacterium glutamicum mdh Gene Promoter Mutants for the Application in L-Lysine Production

In the present disclosure, T311I point mutation was first introduced into the aspartate kinase lysC gene of Corynebacterium glutamicum ATCC13032 strain, with the codon mutated from ACC to ATC, to obtain a SCgL30 strain. In the present disclosure, the mdh gene promoter mutant was further randomly applied to overexpress phosphonopyruvate carboxylase (PPC, NCBI-GeneID: 1019553, NCBI-Protein ID: NP_600799) gene to test its effect on L-lysine production.

The overexpression plasmid was constructed as follows: ppc gene was overexpressed using the P_{mdh}-39 promoter mutant on the basis of pEC-XK99E plasmid skeleton. A promoter mutant fragment was amplified using the corresponding promoter mutant plasmid screened in Example 2 as a template, and mdh-P1/mdh-P2 as primers: a ppc gene fragment was amplified using Corynebacterium glutamicum ATCC 13032 genome as a template, and ppc-1/ppc-2 as primers: a plasmid skeleton was amplified using pEC-XK99E plasmid as a template, and PEC-1/2 as primers. The above-obtained promoter mutant fragment, the ppc gene fragment and the plasmid skeleton fragment were cloned and ligated via Vazyme's One Step Cloning Kit to obtain a pEC-P_{mdh-39}-ppc plasmid. The pEC-XK99E control plasmid and the aforesaid plasmid were transformed into SCgL30 strains, respectively; to obtain a control strain SCgL 30 (pEC-XK99E) and a promoter mutant overexpression strain SCgL30(pEC-P_{mdh-39}-ppc). The sequences of the primers used above are shown in Table 4.

TABLE 4

| Primers | Nucleotide sequence | SEQ ID NO. |
|---|---|---|
| mdh-P1 | CCTGATGCGGTATTTTCTCC | SEQ ID |
| | CGATTCCCCATGATGCCCTA | NO: 64 |
| | AC | |
| mdh-P2 | TCGCGTAAAAAATCAGTCAT | SEQ ID |
| | TAAACAATCCTCAATCCTTG | NO: 65 |
| | TAGG | |
| ppc-1 | ATGACTGATTTTTTACGCGA | SEQ ID |
| | TGAC | NO: 66 |
| ppc-2 | CCAAGCTTGCATGCCTGCAG | SEQ ID |
| | CTAGCCGGAGTTGCGCAGC | NO: 67 |
| PEC-1 | CTGCAGGCATGCAAGCTTGG | SEQ ID |
| | | NO: 56 |
| PEC-2 | GGAGAAAATACCGCATCAGGC | SEQ ID |
| | | NO: 57 |

### (2) Evaluation of L-Lysine Production Capacity of a Promoter Mutant Overexpressed Strain

In order to test the effect of overexpression of ppc gene by P_{mdh}-39 promoter mutant in Corynebacterium glutamicum on L-lysine production by the strain, fermentation tests were carried out on SCgL30 (pEC-XK99E) and SCgL30 (pEC-P_{mdh-39}-ppc), respectively: Ingredients of the fermentation medium was as follows: glucose, 80 g/L: yeast powder, 1 g/L: soybean peptone, 1 g/L: NaCl, 1 g/L: ammonium sulfate, 1 g/L: Urea, 10 g/L: K₂HPO₄·3H₂O, 1 g/L: MgSO₄·7H₂O, 0.45 g/L: FeSO₄·7H₂O, 0.05 g/L: biotin, 0.4 mg/L: vitamin B1, 0.1 mg/L: MOPS, 40 g/L: initial pH7.2. 25 μg/mL Kanamycin was supplemented to the medium. Firstly, the strains were inoculated into TSB liquid medium to undergo culture for 8 hours, the cultures were inoculated as seeds into a 24-well plate containing 800 μl of fermentation medium in each well, and the inoculation amount was 12 μl. The initial OD₆₀₀ was controlled at about 0.1, and the culture was carried out at 30° C. for 17 hours at a rotation speed of the plate shaker of 800 rpm, with three parallel strains for each strain. After the fermentation, the L-lysine yield was detected by SBA biosensor and OD₆₀₀ was measured by a microplate reader. The results are shown in Table 5. The L-lysine yield using mdh promoter mutant overexpression strain SCgL30(pEC-P_{mdh-39}-ppc) was increased by 59%, indicating that the promoter mutants of the present disclosure can be used to enhance the expression of ppc gene and can be applied to L-lysine production.

TABLE 5

| Strains | OD₆₀₀ | L-lysine yield (g/L) |
|---|---|---|
| SCgL30(pEC-XK99E) | 17.7 ± 1.2 | 4.50 ± 0.0 |
| SCgL30(pEC-P_{mdh-39}-ppc) | 18.5 ± 1.0 | 7.17 ± 0.3 |

As shown by the above results, the promoter mutants of the present disclosure can be used to enhance the expression of PPC in Corynebacterium glutamicum, and thereby enhancing the synthesis from phosphoenolpyruvate (PEP) to oxaloacetic acid, thus can be applied to the production of an oxaloacetic acid precursor supply-dependent target product including aspartic acid family amino acids (lysine, threonine, isoleucine, methionine), glutamic acid family amino acids (glutamic acid, proline, hydroxyproline, arginine, glutamine), and to the biological production of amino acids such as 5-aminolevulinic acid for which oxaloacetic acid is an important metabolic precursor.

Since enhancing the expression and the activity of PPC can be used to increase the yield of a target compound such as 5-aminolevulinic acid^{[}1], while all the promoter mutants of the present disclosure can be used to enhance the expression and the activity of PPC, thus the promoter mutants of the present disclosure can also be applied to the production of 5-aminolevulinic acid.

It has been verified that the promoter mutants of the present disclosure can improve the expression of a fusion protein of the N-terminus of Streptococcus mutans derived gapN gene and RFP, and can also be used to improve the expression of PPC, and the promoter mutants of the present disclosure can be also used to express other genes and be applied to the production of various products.

P_{mdh}-1(SEQ ID NO: 1):
cgattccccatgatgccctaacatcttgcaggtgaggggtacata
ttggggcaattcgggggtaattttgcagtatcgtcaagatcaccc
aaaactggtggctgttctcttttaagcgggatagcatgggttctt
agaggaccccctacaaggattgaggattgttta
180 bp coding rcgion (SEQ ID NO: 2) of
N-terminus of gapN gene:
atgacaaaacaatataaaaattatgtcaatggcgagtggaagctt
tcagaaaatgaaattaaaatctacgaaccggccagtggagctga
attgggttcagttccagcaatgagtactgaagaagtagattatgt
ttatgcttcagccaagaaagctcaaccagcttggcgatcactttc
a
P_{mdh}-1(SEQ ID NO: 3):
cgattccccatgatgccctaacatcttgcaggtgaggggtacata
ttggggcaattcgggggtaattttgcagtatcgtcaagatcacc
caaaactggtggctgcaggtaacaactgcgagtacggtgggttct
tagaggaccccctacaaggattgaggattgttta
P_{mdh}-2(SEQ ID NO: 4):
cgattccccatgatgccctaacatcttgcaggtgaggggtacata
ttggggcaattcgggggtaattttgcagtatcgtcaagatcacc
caaaactggtggctgagtctgagggatataattaagatgggttct
tagaggaccccctacaaggattgaggattgttta
P_{mdh}-3(SEQ ID NO: 5):
cgattccccatgatgccctaacatcttgcaggtgaggggtacata
ttggggcaattcgggggtaattttgcagtatcgtcaagatcacc
caaaactggtggctgggtcgtggaggacaagttaagatgggttct
tagaggaccccctacaaggattgaggattgttta
P_{mdh}-4(SEQ ID NO: 6):
cgattccccatgatgccctaacatcttgcaggtgaggggtacata
ttggggcaattcgggggtaattttgcagtatcgtcaagatcacc
caaaactggtggctgctactaccatcgggtgctaaggtgggttct
tagaggaccccctacaaggattgaggattgttta
P_{mdh}-5(SEQ ID NO: 7):
cgattccccatgatgccctaacatcttgcaggtgaggggtacata
ttggggcaattcgggggtaattttgcagtatcgtcaagatcacc
caaaactggtggctggataggtaggcgttaagtaaactgggttct
tagaggaccccctacaaggattgaggattgttta
P_{mdh}-6(SEQ ID NO: 8):
cgattccccatgatgccctaacatcttgcaggtgaggggtacata
ttggggcaattcgggggtaattttgcagtatcgtcaagatcacc
caaaactggtggctgggcaattcgcggggaggtagactgggttct
tagaggaccccctacaaggattgaggattgttta
P_{mdh}-7(SEQ ID NO: 9):
cgattccccatgatgccctaacatcttgcaggtgaggggtacata
ttggggcaattcgggggtaattttgcagtatcgtcaagatcacc
caaaactggtggctgtattgggcgcggcattataggatgggttct
tagaggaccccctacaaggattgaggattgttta
P_{mdh}-8(SEQ ID NO: 10):
cgattccccatgatgccctaacatcttgcaggtgaggggtacata
ttggggcaattcgggggtaattttgcagtatcgtcaagatcacc
caaaactggtggctggggaactgctttggtagtatcatgggttct
tagaggaccccctacaaggattgaggattgttta
P_{mdh}-9(SEQ ID NO: 11):
cgattccccatgatgccctaacatcttgcaggtgaggggtacata
ttggggcaattcgggggtaattttgcagtatcgtcaagatcacc
caaaactggtggctggttcaggtttgggccgctatgctgggttct
tagaggaccccctacaaggattgaggattgttta
P_{mdh}-10(SEQ ID NO: 12):
cgattccccatgatgccctaacatcttgcaggtgaggggtacata
ttggggcaattcgggggtaattttgcagtatcgtcaagatcacc
caaaactggtggctgtgatcggggcgctttggtacggtgggttct
tagaggaccccctacaaggattgaggattgttta
P_{mdh}-11(SEQ ID NO: 13):
cgattccccatgatgccctaacatcttgcaggtgaggggtacata
ttggggcaattcgggggtaattttgcagtatcgtcaagatcacc
caaaactggtggctgtatgaaataaattggagtaggctgggttct
tagaggaccccctacaaggattgaggattgttta
P_{mdh}-12(SEQ ID NO: 14):
cgattccccatgatgccctaacatcttgcaggtgaggggtacata
ttggggcaattcgggggtaattttgcagtatcgtcaagatcacc
caaaactggtggctgaacggtatctttctccatataatgggttct
tagaggaccccctacaaggattgaggattgttta
P_{mdh}-13(SEQ ID NO: 15):
cgattccccatgatgccctaacatcttgcaggtgaggggtacata
ttggggcaattcgggggtaattttgcagtatcgtcaagatcacc
caaaactggtggctgaacggagcgtgtacgactagaatgggttct
tagaggaccccctacaaggattgaggattgttta
P_{mdh}-14(SEQ ID NO: 16):
cgattccccatgatgccctaacatcttgcaggtgaggggtacata
ttggggcaattcgggggtaattttgcagtatcgtcaagatcacc
caaaactggtggctgtgcgcgggtaaacgctgtagagtgggttct
tagaggaccccctacaaggattgaggattgttta
P_{mdh}-15(SEQ ID NO: 17):
cgattccccatgatgccctaacatcttgcaggtgaggggtacata
ttggggcaattcgggggtaattttgcagtatcgtcaagatcacc
caaaactggtggctgttgatgtcgtagcgtagtattatgggttct
tagaggaccccctacaaggattgaggattgttta
P_{mdh}-16(SEQ ID NO: 18):
cgattccccatgatgccctaacatcttgcaggtgaggggtacata
ttggggcaattcgggggtaattttgcagtatcgtcaagatcacc
caaaactggtggctgagctcggactttggtggtaggctgggttct
tagaggaccccctacaaggattgaggattgttta
P_{mdh}-17(SEQ ID NO: 19):
cgattccccatgatgccctaacatcttgcaggtgaggggtacata
ttggggcaattcgggggtaattttgcagtatcgtcaagatcacc
caaaactggtggctgccggcgcattataacgctacaatgggttct
tagaggaccccctacaaggattgaggattgttta
P_{mdh}-18(SEQ ID NO: 20):
cgattccccatgatgccctaacatcttgcaggtgaggggtacata
ttggggcaattcgggggtaattttgcagtatcgtcaagatcacc
caaaactggtggctgcgggggtgtaagtcgagtagactgggttct
tagaggaccccctacaaggattgaggattgttta
P_{mdh}-19(SEQ ID NO: 21):
cgattccccatgatgccctaacatcttgcaggtgaggggtacata
ttggggcaattcgggggtaattttgcagtatcgtcaagatcacc
caaaactggtggctgacgggtaggtatttcgctaggatgggttct
tagaggaccccctacaaggattgaggattgttta
P_{mdh}-20(SEQ ID NO: 22):
cgattccccatgatgccctaacatcttgcaggtgaggggtacata
ttggggcaattcgggggtaattttgcagtatcgtcaagatcacc
caaaactggtggctgtagtgtatgcttttacatagactgggttct
tagaggaccccctacaaggattgaggattgttta
P_{mdh}-21(SEQ ID NO: 23):
cgattccccatgatgccctaacatcttgcaggtgaggggtacata
ttggggcaattcgggggtaattttgcagtatcgtcaagatcacc
caaaactggtggctgaggcagcatgtacatgatatcgtgggttct
tagaggaccccctacaaggattgaggattgttta
P_{mdh}-22(SEQ ID NO: 24):
cgattccccatgatgccctaacatcttgcaggtgaggggtacata
ttggggcaattcgggggtaattttgcagtatcgtcaagatcacc
caaaactggtggctgttgagcggagttaatagtacactgggttct
tagaggaccccctacaaggattgaggattgttta
P_{mdh}-23(SEQ ID NO: 25):
cgattccccatgatgccctaacatcttgcaggtgaggggtacata
ttggggcaattcgggggtaattttgcagtatcgtcaagatcacc
caaaactggtggctgatacacacaactttcgatactatgggttct
tagaggaccccctacaaggattgaggattgttta
P_{mdh}-24(SEQ ID NO: 26):
cgattccccatgatgccctaacatcttgcaggtgaggggtacata
ttggggcaattcgggggtaattttgcagtatcgtcaagatcacc
caaaactggtggctgattccaaccttatgttatactgtgggttct
tagaggaccccctacaaggattgaggattgttta
P_{mdh}-25(SEQ ID NO: 27):
cgattccccatgatgccctaacatcttgcaggtgaggggtacata
ttggggcaattcgggggtaattttgcagtatcgtcaagatcacc
caaaactggtggctggaggtagtcgtttggggtaggatgggttct
tagaggaccccctacaaggattgaggattgttta
P_{mdh}-26(SEQ ID NO: 28):
cgattccccatgatgccctaacatcttgcaggtgaggggtacata
ttggggcaattcgggggtaattttgcagtatcgtcaagatcacc
caaaactggtggctgattgctcgcgtaaatggtaaactgggttct
tagaggaccccctacaaggattgaggattgttta
P_{mdh}-27(SEQ ID NO: 29):
cgattccccatgatgccctaacatcttgcaggtgaggggtacata
ttggggcaattcgggggtaattttgcagtatcgtcaagatcacc
caaaactggtggctgtgttattatgggagatgtaagttgggttct
tagaggaccccctacaaggattgaggattgttta
P_{mdh}-28(SEQ ID NO: 30):
cgattccccatgatgccctaacatcttgcaggtgaggggtacata
ttggggcaattcgggggtaattttgcagtatcgtcaagatcacc
caaaactggtggctggaaaaatgtacgtgtgttaaaatgggttct
tagaggaccccctacaaggattgaggattgttta
P_{mdh}-29(SEQ ID NO: 31):
cgattccccatgatgccctaacatcttgcaggtgaggggtacata
ttggggcaattcgggggtaattttgcagtatcgtcaagatcacc
caaaactggtggctgagtcctcagtggagtggtaacatgggttct
tagaggaccccctacaaggattgaggattgttta
P_{mdh}-30(SEQ ID NO: 32):
cgattccccatgatgccctaacatcttgcaggtgaggggtacata
ttggggcaattcgggggtaattttgcagtatcgtcaagatcacc
caaaactggtggctgtttccggtgttttcgcgtagagtgggttct
tagaggaccccctacaaggattgaggattgttta
P_{mdh}-31(SEQ ID NO: 33):
cgattccccatgatgccctaacatcttgcaggtgaggggtacata
ttggggcaattcgggggtaattttgcagtatcgtcaagatcacc
caaaactggtggctgggcgtcggtaggtatgttattgtgggttct
tagaggaccccctacaaggattgaggattgttta
P_{mdh}-32(SEQ ID NO: 34):
cgattccccatgatgccctaacatcttgcaggtgaggggtacata
ttggggcaattcgggggtaattttgcagtatcgtcaagatcacc
caaaactggtggctgacggcgatcagagtgtgtaagatgggttct
tagaggaccccctacaaggattgaggattgttta
P_{mdh}-33(SEQ ID NO: 35):
cgattccccatgatgccctaacatcttgcaggtgaggggtacata
ttggggcaattcgggggtaattttgcagtatcgtcaagatcacc
caaaactggtggctgccgtgcgtgatatggggtagtgtgggttct
tagaggaccccctacaaggattgaggattgttta
P_{mdh}-34(SEQ ID NO: 36):
cgattccccatgatgccctaacatcttgcaggtgaggggtacata
ttggggcaattcgggggtaattttgcagtatcgtcaagatcacc
caaaactggtggctggggttgtgagttatcggtataatgggttct
tagaggaccccctacaaggattgaggattgttta
P_{mdh}-35(SEQ ID NO: 37):
cgattccccatgatgccctaacatcttgcaggtgaggggtacata
ttggggcaattcgggggtaattttgcagtatcgtcaagatcacc
caaaactggtggctgtgctccgatgatggtggtaagatgggttct
tagaggaccccctacaaggattgaggattgttta
P_{mdh}-36(SEQ ID NO: 38):
cgattccccatgatgccctaacatcttgcaggtgaggggtacata
ttggggcaattcgggggtaattttgcagtatcgtcaagatcacc
caaaactggtggctggttctcacgggtccgtgtatgatgggttct
tagaggaccccctacaaggattgaggattgttta
P_{mdh}-37(SEQ ID NO: 39):
cgattccccatgatgccctaacatcttgcaggtgaggggtacata
ttggggcaattcgggggtaattttgcagtatcgtcaagatcacc
caaaactggtggctgtttcggaatggatggtgtacgctgggttct
tagaggaccccctacaaggattgaggattgttta
P_{mdh}-38(SEQ ID NO: 40):
cgattccccatgatgccctaacatcttgcaggtgaggggtacata
ttggggcaattcgggggtaattttgcagtatcgtcaagatcacc
caaaactggtggctgattatgtcttcttttggtaaaatgggttct
tagaggaccccctacaaggattgaggattgttta
P_{mdh}-39(SEQ ID NO: 41):
cgattccccatgatgccctaacatcttgcaggtgaggggtacata
ttggggcaattcgggggtaattttgcagtatcgtcaagatcacc
caaaactggtggctggatcataggttctgtggtagattgggttct
tagaggaccccctacaaggattgaggattgttta
P_{mdh}-40(SEQ ID NO: 42):
cgattccccatgatgccctaacatcttgcaggtgaggggtacata
ttggggcaattcgggggtaattttgcagtatcgtcaagatcacc
caaaactggtggctgcatggccgatcatgatgtaatatgggttct
tagaggaccccctacaaggattgaggattgttta
P_{mdh}-41(SEQ ID NO: 43):
cgattccccatgatgccctaacatcttgcaggtgaggggtacata
ttggggcaattcgggggtaattttgcagtatcgtcaagatcacc
caaaactggtggctgtagtggcggtgtacgtgtatgatgggttct
tagaggaccccctacaaggattgaggattgttta
P_{mdh}-42(SEQ ID NO: 44):
cgattccccatgatgccctaacatcttgcaggtgaggggtacata
ttggggcaattcgggggtaattttgcagtatcgtcaagatcacc
caaaactggtggctgatttgttacaggtatggtagtttgggttct
tagaggaccccctacaaggattgaggattgttta
P_{mdh}-43(SEQ ID NO: 45):
cgattccccatgatgccctaacatcttgcaggtgaggggtacata
ttggggcaattcgggggtaattttgcagtatcgtcaagatcacc
caaaactggtggctgacggaacttatttctggtaacgtgggttct
tagaggaccccctacaaggattgaggattgttta
P_{mdh}-44(SEQ ID NO: 46):
cgattccccatgatgccctaacatcttgcaggtgaggggtacata
ttggggcaattcgggggtaattttgcagtatcgtcaagatcacc
caaaactggtggctgaccgggaggactaatggtatggtgggttct
tagaggaccccctacaaggattgaggattgttta
P_{mdh}-45(SEQ ID NO: 47):
cgattccccatgatgccctaacatcttgcaggtgaggggtacata
ttggggcaattcgggggtaattttgcagtatcgtcaagatcacc
caaaactggtggctggccacccctttttctgctattatgggttct
tagaggaccccctacaaggattgaggattgttta
P_{mdh}-46(SEQ ID NO: 48):
cgattccccatgatgccctaacatcttgcaggtgaggggtacata
ttggggcaattcgggggtaattttgcagtatcgtcaagatcacc
caaaactggtggctgttgcgccggagttgcggtatagtgggttct
tagaggaccccctacaaggattgaggattgttta
P_{mdh}-47(SEQ ID NO: 49):
cgattccccatgatgccctaacatcttgcaggtgaggggtacata
ttggggcaattcgggggtaattttgcagtatcgtcaagatcacc
caaaactggtggctggtttacgggtgttcatgtagtatgggttct
tagaggaccccctacaaggattgaggattgttta
P_{mdh}-48(SEQ ID NO: 50):
cgattccccatgatgccctaacatcttgcaggtgaggggtacata
ttggggcaattcgggggtaattttgcagtatcgtcaagatcacc
caaaactggtggctgtttgtctcgtatctgtgtaagatgggttct
tagaggaccccctacaaggattgaggattgttta
P_{mdh}-49(SEQ ID NO: 51):
cgattccccatgatgccctaacatcttgcaggtgaggggtacata
ttggggcaattcgggggtaattttgcagtatcgtcaagatcacc
caaaactggtggctgattttgggcacttagtgtattgtgggttct
tagaggaccccctacaaggattgaggattgttta

### References

[1] CN103981203A
[2] Wang, Y C et al., Screening efficient constitutive promoters in Corynebacterium glutamicum based on time-series transcriptome analysis. Chinese Journal of Biotechnology, 2018, 34(11):1760-1771

All technical features disclosed in this specification can be combined in any way of combination. Each feature disclosed in this specification can also be replaced by another feature having the same, equal or similar function. Therefore, unless otherwise specified, each feature disclosed herein is merely an example of a series of equal or similar features. In addition, according to the above description of the present disclosure, those skilled

in the art can easily understand the key features of the present disclosure, and many modifications can be made to the invention to adapt to various use purposes and conditions without departing from the spirit and scope of the present disclosure, and therefore such modifications are also intended to fall within the scope of the appended claims.

1. A polynucleotide having promoter activity, wherein the polynucleotide is selected from the group shown in any one of the following (i)-(iv):
(i) a mutant of a polynucleotide comprising the sequence as set forth in SEQ ID NO: 1, comprising a mutated nucleotide at one or more positions in position 105 to position 126 of the sequence as set forth in SEQ ID NO: 1;
(ii) a polynucleotide comprising a sequence complementary to the nucleotide sequence as set forth in (i);
(iii) a polynucleotide comprising a reversely complementary sequence of a sequence capable of hybridizing with the nucleotide sequence as set forth in (i) or (ii) under high stringent hybridization conditions or very high stringent hybridization conditions;
(iv) a polynucleotide having at least 90%, sequence identity compared with the nucleotide sequence as set forth in (i) or (ii);
wherein the polynucleotide as set forth in any one of (i) to (iv) has a nucleotide sequence that is not TTCTCTTTTAAGCGGGATAGCA at position 105 to position 126 of the sequence as set forth in SEQ ID NO: 1; and the polynucleotide as set forth in any one of (i) to (iv) has enhanced promoter activity compared with the polynucleotide having the sequence as set forth in SEQ ID NO: 1.

2. The polynucleotide having promoter activity according to claim 1, wherein the mutant has 0.4 to 13 folds or more improved promoter activity as compared with the promoter activity of the polynucleotide having the sequence as set forth in SEQ ID NO: 1.

3. The polynucleotide having promoter activity according to claim 1, wherein the nucleotide sequence of the mutant corresponding to position 105 to position 126 of the sequence as set forth in SEQ ID NO: 1 is selected from any one of the group consisting of the following (P_{mdh}-1) to (P_{mdh}-49):
(P_{mdh}-1)
CAGGTAACAACTGCGAGTACGG;
(P_{mdh}-2)
AGTCTGAGGGATATAATTAAGA;
(P_{mdh}-3)
GGTCGTGGAGGACAAGTTAAGA;
(P_{mdh}-4)
CTACTACCATCGGGTGCTAAGG;
(P_{mdh}-5)
GATAGGTAGGCGTTAAGTAAAC;
(P_{mdh}-6)
GGCAATTCGCGGGGAGGTAGAC;
(P_{mdh}-7)
TATTGGGCGCGGCATTATAGGA;
(P_{mdh}-8)
GGGAACTGCTTTGGTAGTATCA;
(P_{mdh}-9)
GTTCAGGTTTGGGCCGCTATGC;
(P_{mdh}-10)
TGATCGGGGCGCTTTGGTACGG;
(P_{mdh}-11)
TATGAAATAAATTGGAGTAGGC;
(P_{mdh}-12)
AACGGTATCTTTCTCCATATAA;
(P_{mdh}-13)
AACGGAGCGTGTACGACTAGAA;
(P_{mdh}-14)
TGCGCGGGTAAACGCTGTAGAG;
(P_{mdh}-15)
TTGATGTCGTAGCGTAGTATTA;
(P_{mdh}-16)
AGCTCGGACTTTGGTGGTAGGC;
(P_{mdh}-17)
CCGGCGCATTATAACGCTACAA;
(P_{mdh}-18)
CGGGGGTGTAAGTCGAGTAGAC;
(P_{mdh}-19)
ACGGGTAGGTATTTCGCTAGGA;
(P_{mdh}-20)
TAGTGTATGCTTTTACATAGAC;
(P_{mdh}-21)
AGGCAGCATGTACATGATATCG;
(P_{mdh}-22)
TTGAGCGGAGTTAATAGTACAC;
(P_{mdh}-23)
ATACACACAACTTTCGATACTA;
(P_{mdh}-24)
ATTCCAACCTTATGTTATACTG;
(P_{mdh}-25)
GAGGTAGTCGTTTGGGGTAGGA;
(P_{mdh}-26)
ATTGCTCGCGTAAATGGTAAAC;
(P_{mdh}-27)
TGTTATTATGGGAGATGTAAGT;
(P_{mdh}-28)
GAAAAATGTACGTGTGTTAAAA;
(P_{mdh}-29)
AGTCCTCAGTGGAGTGGTAACA;
(P_{mdh}-30)
TTTCCGGTGTTTTCGCGTAGAG;
(P_{mdh}-31)
GGCGTCGGTAGGTATGTTATTG;
(P_{mdh}-32)
ACGGCGATCAGAGTGTGTAAGA;
(P_{mdh}-33)
CCGTGCGTGATATGGGGTAGTG;
(P_{mdh}-34)
GGGTTGTGAGTTATCGGTATAA;
(P_{mdh}-35)
TGCTCCGATGATGGTGGTAAGA;
(P_{mdh}-36)
GTTCTCACGGGTCCGTGTATGA;
(P_{mdh}-37)
TTTCGGAATGGATGGTGTACGC;
(P_{mdh}-38)
ATTATGTCTTCTTTTGGTAAAA;
(P_{mdh}-39)
GATCATAGGTTCTGTGGTAGAT;
(P_{mdh}-40)
CATGGCCGATCATGATGTAATA;
(P_{mdh}-41)
TAGTGGCGGTGTACGTGTATGA;
(P_{mdh}-42)
ATTTGTTACAGGTATGGTAGTT;
(P_{mdh}-43)
ACGGAACTTATTTCTGGTAACG;
(P_{mdh}-44)
ACCGGGAGGACTAATGGTATGG;
(P_{mdh}-45)
GCCACCCCTTTTTCTGCTATTA;
(P_{mdh}-46)
TTGCGCCGGAGTTGCGGTATAG;
(P_{mdh}-47)
GTTTACGGGTGTTCATGTAGTA;
(P_{mdh}-48)
TTTGTCTCGTATCTGTGTAAGA;
(P_{mdh}-49)
ATTTTGGGCACTTAGTGTATTG.

4. The polynucleotide having promoter activity according to claim 1, wherein the nucleotide sequence of the mutant is selected from sequences as set forth in any one of SEQ ID NOs: 3 to 51.

5. A transcription expression cassette, comprising the polynucleotide having promoter activity according to claim 1;
optionally, wherein the transcription expression cassette further contains a target gene that is operably ligated to the polynucleotide having promoter activity; the target gene is a protein coding gene.

6. A recombinant expression vector comprising the polynucleotide having promoter activity according to claim 1.

7. A recombinant host cell comprising the transcription expression cassette according to claim 5.

8. The recombinant host cell according to claim 7, wherein the host cell is from genus Corynebacterium, genus Brevibacterium, genus Arthrobacterium, genus Microbacterium or genus Escherichia.

9-11. (canceled)

12. A construction method of a promoter mutant, including the following steps:
mutation step: mutating a polynucleotide having the sequence as set forth in SEQ ID NO: 1 so that there are mutated nucleotides at one or more positions in position 105 to position 126 of the sequence as set forth in SEQ ID NO: 1;
screening step: screening for a mutant of a polynucleotide having improved promoter activity as compared with the polynucleotide having the sequence as set forth in SEQ ID NO: 1, to obtain a promoter mutant.

13. The construction method according to claim 12, wherein the mutation step includes:
mutating the polynucleotide having the sequence as set forth in SEQ ID NO: 1 so that the nucleotides at position 105 to position 126 of the sequence as set forth in SEQ ID NO: 1 are mutated to a nucleotide sequence as set forth below: NNNNNNNNNNNNNNNNNTANNNT;
wherein N is selected from A, T, C or G;
wherein the promoter mutant has 0.4 to 13 folds or more improved promoter activity as compared with the promoter activity of the polynucleotide having the sequence as set forth in SEQ ID NO: 1.

14. A method for regulating transcription, wherein the method includes a step of operably ligating the polynucleotide having promoter activity according to claim 1 to a target RNA or a target gene; wherein the target RNA includes at least one of tRNA, sRNA, the target gene includes at least one of a coding gene of a protein related to the synthesis of a target compound, a coding gene of a gene expression regulatory protein and a coding gene of a protein related to membrane transport.

15. A method for preparing a protein, wherein the method includes a step of expressing the protein by using the transcription expression cassette according to claim 5, in which the protein is a protein related to the synthesis of a target compound, or a gene expression regulatory protein; and
the method further includes a step of isolating or purifying the protein.

16. A method for producing a target compound, wherein the method includes a step of expressing a protein related to the synthesis of a target compound, a protein related to membrane transport, or a gene expression regulatory protein by using the transcription expression cassette according to claim 5, and producing the target compound in the presence of the protein associated with the synthesis of the target compound, a protein related to membrane transport, or the gene expression regulatory protein; and
the method further includes a step of isolating or purifying the target compound.

17. The recombinant host cell according to claim 8, wherein the host cell is Corynebacterium glutamicum or Escherichia coli.

18. The recombinant host cell according to claim 17, wherein the host cell is Corynebacterium glutamicum ATCC 13032, Corynebacterium glutamicum ATCC 13869, Corynebacterium glutamicum ATCC 14067 or derivative strains of Corynebacterium glutamicum.

19. The method according to claim 14, wherein the target gene includes at least one of the following genes: pyruvate carboxylase gene, phosphoenolpyruvate carboxylase gene, γ-glutamyl kinase gene, glutamate semialdehyde dehydrogenase gene, pyrroline-5-carboxylate eductase gene, amino acid transport protein gene, ptsG system related gene, pyruvate dehydrogenase gene, homoserine dehydrogenase gene, oxaloacetic acid decarboxylase gene, gluconic acid repressor gene, glucose dehydrogenase gene, aspartate kinase gene, aspartate semialdehyde dehydrogenase gene, aspartate ammonia lyase gene, dihydrodipicolinate synthase gene, dihydropyridinic acid reductase gene, succinyl diaminopimelate aminotransferase gene, tetrahydrodipicolinate succinylase gene, succinyl diaminopimelate deacylase gene, diaminopimelic acid epimerase gene, diaminopimelic acid deacylase gene, glyceraldehyde-3-phosphate dehydrogenase gene, transketolase gene, diaminopimelate dehydrogenase gene.

20. The method according to claim 16, wherein the target compound includes at least one of amino acids and organic acids;
the amino acids include one or a combination of two or more of the following: lysine, glutamic acid, threonine, proline, hydroxyproline, glycine, alanine, valine, leucine, isoleucine, serine, cysteine, glutamine, methionine, aspartic acid, asparagine, arginine, histidine, phenylalanine, tyrosine, tryptophan, 5-aminolevulinic acid or derivatives of any one of the above amino acids;
the organic acids include one or a combination of two or more of the following: citric acid, succinic acid, lactic acid, acetic acid, butyric acid, palmitic acid, oxalic acid, oxaloacetic acid, tartaric acid, propionic acid, hexenoic acid, decanoic acid, caprylic acid, valeric acid, malic acid or derivatives of any one of the above organic acids.

21. The method according to claim 16, wherein the protein related to the synthesis of the target compound is a protein related to the synthesis of an L-amino acid; the protein related to the synthesis of an L-amino acid includes one or a combination of two or more of: pyruvate carboxylase, phosphoenolpyruvate carboxylase, γ-glutamyl kinase, glutamate semialdehyde dehydrogenase, pyrroline-5-carboxylate reductase, amino acid transport protein, ptsG system, pyruvate dehydrogenase, homoserine dehydrogenase, oxaloacetic acid decarboxylase, gluconic acid repressor, glucose dehydrogenase, aspartate kinase, aspartate semialdehyde dehydrogenase, aspartate ammonia lyase, dihydrodipicolinate synthase, dihydropyridinic acid reductase, succinyl diaminopimelate aminotransferase, tetrahydrodipicolinate succinylase, succinyl diaminopimelate deacylase, diaminopimelic acid epimerase, diaminopimelic acid deacylase, glyceraldehyde-3-phosphate dehydrogenase, transketolase, diaminopimelate dehydrogenase.
